(19) United States
(12) Patent Application Publication
(10) Pub. No.: US 2019/0302102 A1
(43) Pub. Date: Oct. 3, 2019

(54) COMBINATION THERAPIES FOR TREATING BIPOLAR DISORDER, AND METHODS FOR USING THE SAME

(71) Applicant: PsychNostics, LLC, Baltimore, MD (US)

(72) Inventor: Alagu P. THIRUVENGADAM, Baltimore, MD (US)

(73) Assignee: PsychNostics, LLC, Baltimore, MD (US)

(21) Appl. No.: 16/346,212

(22) PCT Filed: Oct. 26, 2017
(86) PCT No.: PCT/US2017/058465

§ 371 Date: Apr. 30, 2019

Related U.S. Application Data
(60) Provisional application No. 62/420,934, filed on Nov. 11, 2016.

(51) Int. Cl. G01N 33/50; A61K 33/06; A61K 45/06; G16H 20/10

(52) U.S. Cl. CPC G01N 33/5044; A61K 33/06; G01N 2800/52; G16H 20/10; G01N 2800/304; A61K 45/06

(57) ABSTRACT

The present invention relates to pharmaceutical combinations and compositions, and methods of using the same for treatment of Bipolar Disorder (BD). More specifically, the invention relates to combination therapies for the treatment of BD, and methods for treating BD using such therapies. The present invention also relates to methods of determining an optimal combination drug treatment therapy for BD, methods of optimizing a combination drug treatment therapy for BD, methods of optimizing dosage of a drug in a combination drug treatment therapy for BD, as well as methods for monitoring the efficacy of a combination therapy for the treatment of BD. The present invention involves analyzing the membrane potential of cells isolated from a BD patient treated with the combination therapy, and calculating a membrane potential ratio therefrom.

### FIELD OF THE INVENTION

The present invention relates to the treatment of Bipolar Disorder (BD), and more specifically, to combination therapies for the treatment of BD, and methods for treating BD using such therapies. The present invention relates to a method for optimizing drug therapy treatment for BD and a method of optimizing drug dosage for treatment of BD. These methods include optimization of a combination therapy for treatment of BD, and optimization of a drug dosage in a combination therapy for treatment of BD. The methods of the present invention involve analyzing the membrane potential of cells isolated from a BD patient, and calculating a membrane potential ratio therefrom. The present invention further relates to increasing the therapeutic efficacy of a drug therapy treatment for BD as well as monitoring the efficacy of a combination therapy for the treatment of BD, by analyzing the membrane potential of cells isolated from a BD patient treated with the combination therapy, and calculating a membrane potential ratio therefrom.

### BACKGROUND OF THE INVENTION

Mental illness afflicts nearly ten percent of the general population both in the United States and in the rest of the world. Bipolar (manic depressive) disorder occurs in one to two percent of the population, and is the sixth leading cause of disability (Coryell et al., Am. J. Psychiatry 150:720-727 (1993); Lopez et al., Nat. Med. 4:1241-1243 (1998); Hyman, S. E., Am. J. Geriatr. Psychiatry 9:330-339 (2001)). A problem facing the medical community is misdiagnosis of bipolar disorder. Misdiagnosed patients receive an average of 3.5 misdiagnoses and consult four physicians before receiving an accurate diagnosis (“Living with bipolar disorder. How far have we really come?” National Depressive and Manic-Depressive Association. Chicago, Ill. (2001)).

BD is one of the major mental illnesses difficult to diagnose and to treat. Even though Cade (1) discovered the mood stabilizing properties of lithium in BD patients during the mid 1900s, the mechanism of action of lithium in BD is still controversial (Goodwin and Jamison (2); Manji. Bowden and Belmaker (3), and Fieve (19)). However Schou (4) conducted extensive clinical trials and established lithium's mood stabilizing power in BD patients. Lithium is the only clinically proven mood stabilizer used to treat BD (2, 3). Its toxic level is about 2 mM whereas its therapeutic level is around 1.2 mM. The side effects at this level include nausea, diarrhea, dizziness, muscle weakness, fatigue, and a dazed feeling. These unwanted side effects often improve with continued use. Fine tremor, frequent urination, and thirst can occur and may persist with continued use. Weight gain and swelling from excess fluid can also occur. Periodic Blood tests are required. All these symptoms are dosage dependant. Patients' tolerance and compliance at high therapeutic levels are limited. Lithium is the only clinically-proven mood stabilizer used to treat bipolar disorder. (Goodwin et al., Manic-Depressive Illness. Oxford University Press, 2007; Goodwin et al., “The impact of the discovery of lithium on psychiatric thought and practice in the USA and Europe.” Australian and New Zealand Journal of Psychiatry, 1999, 33: S54-S64; Manji et al., Bipolar Medications-Mechanisms of Action, American psychiatric Press, Washington D.C. 2000). Schou (“The early European lithium studies,” Australian and New Zealand Journal of Psychiatry, 1999, 33: S39-S47) conducted extensive clinical trials and established lithium's mood stabilizing power in BD patients. However, the concentration at which it is generally recognized as being therapeutic (around 1.2 mM) is close to the concentration at which it is toxic (about 2 mM). Thus, since the therapeutic concentration is so close to the concentration at which it is toxic, lithium often causes severe side effects that are not well tolerated by patients. For example, even at the therapeutic concentration of 1.2 mM, side effects may result including nausea, diarrhea, dizziness, muscle weakness, fatigue, and a dazed feeling. Although these unwanted side effects often improve with continued use, fine tremor, frequent urination, and thirst can occur and may persist even with continued use. Weight gain and swelling from excess fluid may also occur from continued use. Because of this battery of side effects, lithium is often poorly tolerated by BD patients, and compliance at high therapeutic levels is limited. Additionally, to balance efficacy with the goal of minimizing side effects, frequent blood tests are required to ensure that the lithium concentration in BD patients remains at a therapeutic, but below toxic, concentration. These side effects, however, are dose-dependent. These findings highlight the persistent and chronic nature of bipolar disorder as well as the magnitude of unmet needs in its treatment.

### SUMMARY OF THE INVENTION

The present invention relates to the fields of clinical psychiatry, clinical psychology and more specifically to the treatment of patients with BD using combination therapies. The present invention also relates to determining the optimum dose of a combination therapy for the treatment of BD, by analyzing the membrane potential of cells isolated from a BD patient treated with the combination therapy, and calculating a membrane potential ratio therefrom. The present invention further relates to monitoring the efficacy of a combination therapy for the treatment of BD, by analyzing the membrane potential of cells isolated from a BD patient treated with the combination therapy, and calculating a membrane potential ratio therefrom.

In one aspect, the present invention provides a method of determining an optimal combination drug treatment therapy for a patient with bipolar disorder (BD), that comprises obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K⁺ or absence of K⁺; comparing the ratio of the mean membrane potential to (a) and/or (b) (a) a control ratio of a mean membrane potential of first population of control human cells known to not have BD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+, (b) a bipolar control ratio of a mean membrane potential of first population of bipolar control human cells known to have BD incubated in vitro in the presence of the agent that alters diacylglycerol signaling and in the absence of K+, to a mean membrane potential of a second population of the bipolar control human cells incubated in vitro in the absence of the agent that alters diacylglycerol signaling and in the presence of K+ or absence of K+; and identifying the optimal combination drug treatment therapy when the ratio of the mean membrane potential obtained is not significantly different from the control ratio of (a), is increased towards the control ratio (a) in comparison to or relative to the bipolar control ratio of (b), and/or is higher in comparison to or relative to the BD control ratio of (b).

In a second aspect, the present invention provides a method of optimizing a combination drug treatment therapy for a patient with bipolar disorder (BD), comprising the steps of: obtaining at least one sample from a BD patient in a drug therapy treatment for BD; performing on each sample, a mean membrane potential test comprising obtaining a ratio of a mean membrane potential that is a mean membrane potential of a first population of cells from the sample incubated in vitro in the presence of an agent that alters diacylglycerol signaling and in the absence of K⁺, to a mean membrane potential of a second population of the sample incubated in vitro in the absence of the test agent that alters diacylglycerol signaling and in the presence of K⁺ or absence of K⁺; comparing the ratio of the mean membrane potential to (a) and/or (b):(a) a control ratio of a mean membrane potential of a first population of control human cells known to not have BD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+, (b) a bipolar control ratio of a mean membrane potential of a first population of bipolar control human cells known to have BD incubated in vitro in the presence of the agent that alters diacylglycerol signaling and in the absence of K+, to a mean membrane potential of a second population of the bipolar control human cells incubated in vitro in the absence of the agent that alters diacylglycerol signaling and in the presence of K+ or absence of K+; determining an optimal drug therapy treatment for the BD patient when the ratio of the mean membrane potential obtained is not significantly different from the control ratio of (a), is increased towards the control ratio (a) in comparison to or relative to the bipolar control ratio of (b), and/or is higher in comparison to or relative to the BD control ratio of (b). The method may further include optionally, modifying at least one drug in the drug therapy treatment for BD when the least one drug treatment therapy for BD is determined to not be the optimal drug treatment therapy for the BD patient based on the mean membrane potential. For instance, such as when the ratio of the mean membrane potential obtained is lower in comparison to or relative to the control ratio of (a), is decreased towards the bipolar control ratio of (b) in comparison to or relative to the control ratio of (a), and/or is not significantly different from the BD control ratio of (b).

In a third aspect, the present invention provides a method for determining an optimum dosage of at least one drug in a combination drug treatment therapy for the treatment of bipolar disorder (BD), said method comprising: obtaining at least one sample from a BD patient treated with a dosage of a drug in a combination therapy; performing on each sample, a mean membrane potential test comprising: obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K⁺ or absence of K⁺; comparing the ratio of the mean membrane potential to (a) and/or (b):(a) a control ratio of a mean membrane potential of a first population of cells from a control human known to not have said BD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+, (b) a bipolar control ratio of a mean membrane potential of a first population of cells from a bipolar control human known to have said BD incubated in vitro in the presence of the agent that alters diacylglycerol signaling and in the absence of K+, to a mean membrane potential of a second population of cells from the bipolar control human incubated in vitro in the absence of the agent that alters diacylglycerol signaling and in the presence of K+ or absence of K+; determining the dosage of the at least one drug in the combination drug treatment therapy is an optimal dosage for treating BD in the combination therapy when the ratio of the mean membrane potential obtained is not significantly different from the control ratio of (a), is increased towards the control ratio (a) in comparison to or relative to the bipolar control ratio of (h), and/or is higher in comparison to or relative to the BD control ratio of (b), or determining the dosage of the drug in the combination drug treatment therapy is not the optimal dosage for treating BD in the combination therapy based on the mean membrane potential. For instance, such as when the ratio of the mean membrane potential obtained is significantly lower in comparison to or relative to the control ratio of (a), is decreased towards the bipolar control ratio of (b) in comparison to or relative to the control ratio of (a), and/or is not significantly different from the BD control ratio of (b). The method may further include optionally, modifying the dosage of the drug in the combination drug treatment therapy when the dosage of the at least one drug in the combination therapy is determined to be not the optimal dosage for treating BD based on the mean membrane potential test.

In a fourth aspect, the present invention provides a method for monitoring the efficacy of a combination drug treatment therapy for the treatment of bipolar disorder (BD), said method comprising: obtaining at least one sample from a BD patient treated with a combination drug treatment therapy for treating BD; performing on each sample, a mean membrane potential test comprising: obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K⁺ or absence of K⁺; comparing the ratio of the mean membrane potential to (a) and/or (b): (a) a control ratio of a mean membrane potential of a first population of cells from a control human known to not have said BD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+. (b) a bipolar control ratio of a mean membrane potential of a first population of cells from a bipolar control human known to have said BD incubated in vitro in the presence of the agent that alters diacylglycerol signaling and in the absence of K+, to a mean membrane potential of a second population of cells from the bipolar control human incubated in vitro in the absence of the agent that alters diacylglycerol signaling and in the presence of K+ or absence of K+; determining the combination drug treatment therapy is efficacious based on the mean membrane potential test when the ratio of the mean membrane potential obtained is not significantly different from the control ratio of (a), is increased towards the control ratio (a) in comparison to or relative to the bipolar control ratio of (b), and/or is significantly higher in comparison to or relative to the BD control ratio of (b), or determining the combination drug treatment therapy is not efficacious based on the mean membrane potential test. For instance, such as when the ratio of the mean membrane potential obtained is lower in comparison to or relative to the control ratio of (a), is decreased towards the bipolar control ratio of (b) in comparison to or relative to the control ratio of (a), and/or is not significantly different from the BD control ratio of (b). The method may further include optionally, adjusting a dosage of one or more agents in the combination drug treatment therapy when the combination therapy is determined to be not efficacious based on the mean membrane potential test.

In the methods described herein, the present invention may further include obtaining an initial ratio of a mean membrane potential from an initial population of cells from the human patient before the obtaining step.

The human cells useful in the present methods may be selected from the group consisting of red blood cells, lymphoblasts, crythocytes, platelets, leukocytes, macrophages, monocytes, dendritic cells, fibroblasts, epidermal cells, mucosal tissue cells, cells of cerebrospinal fluid, hair cells, and whole blood cells.

In a preferred embodiment, the human cells is selected from the group consisting of red blood cells and lymphoblasts.

The combination drug treatment therapy useful in the present methods is a synergistic combination.

The combination drug treatment therapy may comprise a lithium compound and at least one adjunctive agent.

The lithium compound may be present in an effective amount sufficient to provide a plasma lithium level of 1 mM or less. In a preferred embodiment, plasma lithium level is 0.5 mM.

The at least one adjunctive agent useful in the present methods may be selected from the group consisting of a mood stabilizer, an anticonvulsant, an antipsychotic, an anxiolytic, and a cholinergic agonist.

Such cholinergic agonist may be selected from the group consisting of donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof.

Such mood stabilizer may be selected from the group consisting of valproate, divalproex, carbamazepine, lamotrigine, oxcarbazepine, and pharmaceutically acceptable salts thereof.

Such anticonvulsant may be selected from the group consisting of lamotrigine, perampanel, mephobarbital, primidone, phenobarbital, diazepam, clonazepam, lorazepam, clobazam, felbamate, topiramate, acetazolamide, zonisamide, rufinamide, oxcarbazepine, carbamazepine, eslicarbazepine, valproic acid, divalproex sodium, gabapentin, gabapentin enacarbil, tiagabine, phenytoin, fosphenytoin, mephenytoin, ethotoin, magnesium sulfate, lacosamide, ezogabine, trimethadione, levetiracetam, ethosuximide, methsuximide, and pharmaceutically acceptable salts thereof.

Such antipsychotic may be selected from the group consisting of haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof.

Such antidepressant may be selected from the group consisting of fluoxetine, ariprazole, doxepin, clomipramine, bupropion, amoxapine, nortriptyline, vortioxetine, citalopram, duloxetine, trazodone, venlafaxine, selegiline, perphenazine, amitriptyline, levomilnacipram, desvenlafaxine, lurasidone, lamotrigine, escitalopram, chlordiazepoxide, isocarboxazid, phenelzine, desipramine, trazodone, tranylcypromine, paroxetine, mirtazapine, quetiapine, nefazodone, doxepin, trimipramine, imipramine, vilazodone, protriptyline, sertraline, olanzapine, and pharmaceutically acceptable salts thereof.

Such anxiolytic may be selected from the group consisting of secobarbital, mephobarbital, pentobarbital, phenobarbital, amobarbital, butabarbital, estazolam, alprazolam, flurazepam, diazepam, chlordiazepoxide, clorazepate, clonazepam, oxazepam, diazepam, triazolam, lorazepam, temazepam, midazolam, clobazam, diphenhydramine, zolpidem, chloral hydrate, doxepin, sodium oxybate, doxylamine, doxepin, hydroxyzine, meprobamate, ethchlorvynol, eszopiclone, buspirone, zalephon, ramelteon, suvorexant, tryptophan, tasimelteon, dexmedetomidine, and pharmaceutically acceptable salts thereof.

The agent that alters diacylglycerol signaling of the present methods may be selected from the group consisting of a calcium-calmodulin (Ca²⁺/CaM) kinase inhibitor, a diacylglycerol kinase inhibitor, a protein kinase C inhibitor, and an agent that affects calcium-activated potassium (CaK) channels. In a preferred embodiment, the agent is a calcium-calmodulin (Ca²⁺/CaM) kinase inhibitor such as autocamtide-2-related inhibitory peptide (AIP). In another preferred embodiment, the agent is a diacylglycerol kinase inhibitor, such as 6-[2-[4-[(4-fluorophenyl)phenylmethylene]-1-piperidinyl]ethyl]-7-methyl-5H-thiazolo[3,2-alpyrimidin-5-one (ALX).

The mean membrane potential test of the present methods may further include incubating the cells in vitro in buffer comprising a potential-sensitive dye, resuspending the cells in potential-sensitive dye free-buffer, and measuring the cell fluorescence.

The agent that alters K⁺ channel activity of the present methods may be ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

In another aspect, the present invention provides a method of treating bipolar disorder (BD), comprising administering an effective amount of a lithium compound and at least one adjunctive agent to a human patient with BD.

The at least one adjunctive agent and the lithium compound of the method may form a synergistic combination or composition to treat said BD.

The effective amount of lithium compound of the method may be a dose amount that is less than a dosage of lithium required to provide a therapeutically efficacious plasma lithium level for BD therapy when used alone.

In a preferred embodiment, the dose provides a plasma lithium level of 1 mM or less. In another preferred embodiment, the dose provides a plasma lithium level of 0.5 mM or less.

The at least one adjunctive agent of the method may be administered at a dose that is less than a dosage of the at least one adjunctive agent required to provide a therapeutically efficacious plasma level of the at least one adjunctive agent when administered alone.

The at least one adjunctive agent of the method may be selected from the group consisting of a mood stabilizer, an anticonvulsant, an antipsychotic, an anxiolytic, and a cholinergic agonist.

The cholinergic agonist may be selected from the group consisting of donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof. Preferably, the cholinergic agonist is carbachol and pharmaceutically acceptable salts thereof, or donepezil and pharmaceutically acceptable salts thereof.

The antipsychotic may be selected from the group consisting of haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof. Preferably, the antipsychotic is clozapine and pharmaceutically acceptable salts thereof.

In a preferred embodiment, carbachol may be administered to said patient to provide a plasma concentration of 10 μM or less. In another preferred embodiment, donepezil may be administered to said patient to provide a plasma concentration of 10 ng/ml or less. In yet another preferred embodiment, clozapine may be administered to said patient to provide a plasma concentration of 100 ng/ml or less.

In yet another aspect, the present invention provides a method of increasing the therapeutic efficacy of a lithium compound for the treatment of bipolar disorder (BD), comprising administering an effective amount of a lithium compound with at least one adjunctive agent, to a human patient with BD.

The at least one adjunctive agent and the lithium compound of the method may form a synergistic combination or composition to treat said BD.

The effective amount of lithium compound of the method may be a dose amount that is less than a dosage of lithium required to provide a therapeutically efficacious plasma lithium level for BD therapy when used alone.

In a preferred embodiment, the dose provides a plasma lithium level of 1 mM or less. In another preferred embodiment, the dose provides a plasma lithium level of 0.5 mM or less.

The at least one adjunctive agent of the method may be administered at a dose that is less than a dosage of the at least one adjunctive agent required to provide a therapeutically efficacious plasma level of the at least one adjunctive agent when administered alone.

The at least one adjunctive agent of the method may be selected from the group consisting of a mood stabilizer, an anticonvulsant, an antipsychotic, an anxiolytic, and a cholinergic agonist.

The cholinergic agonist may be selected from the group consisting of donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof. Preferably, the cholinergic agonist is carbachol and pharmaceutically acceptable salts thereof, or donepezil and pharmaceutically acceptable salts thereof.

The antipsychotic may be selected from the group consisting of haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof. Preferably, the antipsychotic is clozapine and pharmaceutically acceptable salts thereof.

In a preferred embodiment, carbachol may be administered to said patient to provide a plasma concentration of 10 μM or less. In another preferred embodiment, donepezil may be administered to said patient to provide a plasma concentration of 10 ng/ml or less. In yet another preferred embodiment, clozapine may be administered to said patient to provide a plasma concentration of 100 ng/ml or less.

In another aspect, the present invention provides a pharmaceutical combination comprising a lithium compound and at least one adjunctive agent.

In a further aspect, the present invention provides a pharmaceutical composition comprising a lithium compound and at least one adjunctive agent; and a pharmaceutically acceptable carrier.

The effective amount of lithium compound of the present pharmaceutical combination or composition may be a dose amount that is less than a dosage of lithium required to provide a therapeutically efficacious plasma lithium level for BD therapy when used alone. In a preferred embodiment, the dose provides a plasma lithium level of 1 mM or less. In another preferred embodiment, the dose provides a plasma lithium level of 0.5 mM or less.

The at least one adjunctive agent of the present pharmaceutical combination or composition may be administered at a dose that is less than a dosage of the at least one adjunctive agent required to provide a therapeutically efficacious plasma level of the at least one adjunctive agent when administered alone.

The at least one adjunctive agent may be selected from the group consisting of a mood stabilizer, an anticonvulsant, an antipsychotic, an anxiolytic, and a cholinergic agonist.

The cholinergic agonist may be selected from the group consisting of donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof. In one preferred embodiment, the cholinergic agonist is carbachol and pharmaceutically acceptable salts thereof, or donepezil and pharmaceutically acceptable salts thereof.

The antipsychotic may be selected from the group consisting of haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof. In a preferred embodiment, the antipsychotic is clozapine and pharmaceutically acceptable salts thereof.

The carbachol in the present pharmaceutical combination or composition is administered to said patient to provide a plasma concentration of 10 μM or less.

The donepezil in the present pharmaceutical combination or composition is administered to said patient to provide a plasma concentration of 10 ng/ml or less.

The clozapine in the present pharmaceutical combination or composition is administered to said patient to provide a plasma concentration of 100 ng/ml or less.

Kits of the present invention are provided comprising (a) a K⁺-containing HEPES reference buffer, (b) a K⁺-free HEPES buffer; and (c) a potential-sensitive dye. The kits further include respectively, instructions for performing an assay to determine an optimal combination drug treatment therapy for bipolar disorder, instructions for performing an assay to optimize a combination drug treatment therapy for bipolar disorder, instructions for performing an assay to determine an optimum dosage of a drug in combination drug treatment therapy for bipolar disorder, and instructions for performing an assay to monitor the efficacy of a combination drug treatment therapy for bipolar disorder.

### BRIEF DESCRIPTION OF THE DRAWINGS

FIG. 1 depicts a comparison of the performance of 1 mM Li, with that of 0.5 mM Li+2.5 μM inositol+10 μM carbachol, using the MPR™ test. The synergistic combination of 0.5 mM lithium with carbachol yielded a higher mean MPR™ value of 0.860, as compared to just 0.814 with 1 mM Li alone.

FIG. 2 depicts a comparison of the performance of 1 mM Li, with that of 0.5 mM Li+2.5 μM inositol+100 ng/ml clozapine, using the MPR™ test. The synergistic combination of 0.5 mM lithium with clozapine yielded a higher mean MPR™ value of 0.804, as compared to just 0.757 with 1 mM Li alone.

FIG. 3 depicts a comparison of the performance of 1 mM Li, with that of 0.5 mM Li+2.5 μM inositol+10 ng/ml donepezil, using the MPR™ test. The synergistic combination of 0.5 mM lithium with donepezil yielded a higher mean MPR™ value of 0.796, as compared to just 0.780 with 1 mM Li alone.

FIG. 4 depicts MPR™ returning to negative with treatment. The negative range is approximately below 20 percent of the diagnostic probability.

### DETAILED DESCRIPTION OF THE INVENTION

The present invention relates to the treatment of Bipolar Disorder (BD), and more specifically, to combination therapies for the treatment of BD, and methods for treating BD using such therapies. The present invention also relates to determining the optimum dose of a combination therapy for the treatment of BD, by analyzing the membrane potential of cells isolated from a BD patient treated with the combination therapy, and calculating a membrane potential ratio therefrom. The present invention further relates to monitoring the efficacy of a combination therapy for the treatment of BD, by analyzing the membrane potential of cells isolated from a BD patient treated with the combination therapy, and calculating a membrane potential ratio therefrom.

In some aspects, the present invention relates to combination therapies for the treatment of BD. In preferred embodiments thereof, the combination therapy contains lithium and at least one cholinergic agonist.

As noted above, most biological cells are enclosed by a semi-permeable lipid bilayer that is electrically charged. The electrical voltage across the membrane is called the membrane potential (MP). This potential arises from the ionic gradients between the interior concentrations of ions and the exterior concentration of ions. El Mallakh et al. (“Leukocyte transmembrane potential in bipolar illness,” J. Affect. Disord., 1996, 41: 33-37; the disclosure of which is incorporated herein by reference in its entirety) measured the MP of white blood cells drawn from the blood of hospitalized BD patients, euthymic patients on lithium, and matched non-psychiatric controls. They found that the MP of hospitalized BD patients was hyperpolarized compared to the controls. The MP of cells from euthymic patients was slightly depolarized. Around the same time, the present inventor independently calculated the effect of lithium on MP using the Goldman-Hodgkin-Katz equation for multiple ions, and found that lithium should depolarize the MP. Thiruvengadam (“Effect of lithium and sodium valproate ions on resting membrane potentials in neurons: an hypothesis,” J. Affect. Disord., 2001, 65: 95-99; the disclosure of which is incorporated herein by reference in its entirety) and Thiruvengadam (The Recent Studies On The Electrobiochemical Aspects Of Bipolar Disorder. In: Brown, M. R. (Ed.), Focus on Bipolar Disorder Research. Nova Science Publishers, New York, 2004, pp. 15-35; the disclosure of which is incorporated herein by reference in its entirety).

The present inventor developed a ratiometric assay to measure the ratio of the membrane potential called Membrane Potential Ratio (MPR™), using a reference buffer and a test buffer. Thiruvengadam et al. (“Evaluating the validity of blood-based membrane potential changes for the identification of bipolar disorder,”I. J. Affect Disord., 2007, 100(1-3): 75-82, the disclosure of which is incorporated herein by reference in its entirety). The reference buffer may contain NaCl, CaCl₂ and glucose at physiological concentrations. The buffering agent HEPES was also added to the buffer to maintain the pH. The test buffer may contain ethyl alcohol, preferably, 30% of ethyl alcohol, in addition to the chemicals contained in the reference buffer. The test buffer may contain K⁺ or no K⁺. The membrane potentials were measured in both the buffers and the ratio of the MP in the test buffer to the MP in the reference buffer was designated the “Membrane Potential Ratio” (MPR™). Preferably, both the test buffer and the reference buffer contains no K⁺.

The first clinical trial using MPR™ was carried out at the University Of Maryland School Of Medicine. Hospitalized patients were interviewed by the attending psychiatric department faculty and staff and blood was drawn after their diagnostic evaluation. The final validation was made by the attending faculty using the treatment response of the patients. Thiruvengadam et al. (“Evaluating the validity of blood-based membrane potential changes for the identification of bipolar disorder.” I. J. Affect Disord., 2007, 100(1-3): 75-82, the disclosure of which is incorporated herein by reference in its entirety). In order to cover a broader patient population, a second clinical trial was carried out with the participation of several clinical psychiatrists serving the community. These clinical trials showed that the bipolar group and the ADHD group are significantly different from each other in terms of their MPR™ values.

In the present invention, it was found that the MPR™ (responds to lithium treatment in BD patients and may serve as a validation of the MPR™ test.]

In a biological signaling pathway relevant to MPR™, diacylglycerol (DAG) functions as a second messenger signaling lipid. DAG is a product of the hydrolysis of the phospholipid PIP2 (phosphatidyl inositol-bisphosphate) by the enzyme phospholipase C (PLC, a membrane-bound enzyme). It produces inositol trisphosphate (IP3) through the same reaction. Although inositol trisphosphate (IP3) diffuses into the cytosol, diacylglycerol (DAG) remains within the plasma membrane due to its hydrophobic properties. The production of DAG in the membrane facilitates translocation of PKC from the cytosol to the plasma membrane. Newton (“Protein Kinase C: Poised to signal,” Am. J. Physiol. Endocrinol. Metab., 2010, 298:E395-E402). Hence, both DAG and PKC enzyme play important roles in several signal transduction cascades. Nishizuka Y (“The role of protein kinase C in cell surface signal transduction and tumour promotion,” Nature, 1984, 308(5961): 693-8). Thiruvengadam identified the modulators of the MPRs of patients' cells that could serve as the drug targets for increasing the MPR values in BD patients to the level of the MPR values of Negatives. and identified the DAG signaling pathway as a signaling mechanism that modulates the MPR values.

Phosphatidylinositol 4,5-bisphosphate (PIP2) hydrolysis and diacylglycerol (DAG) signaling are coupled together in producing the therapeutic effects of lithium. Hokin showed that lithium, at concentrations as low as 1 mM (which is a therapeutic plasma concentration for the treatment of bipolar disorder), increased the accumulation of inositol trisphosphate (IP3) in slices of cerebral cortex of guinea pig, rabbit and monkey (a therapeutically relevant model for humans). Hokin LE (“Lithium increases accumulation of second messenger inositol 1,4,5-triphosphate in brain cortex slices in species ranging from mouse to monkey,” Advanced Enzyme Regul., 1993, 33: 299-312). Since DAG is another product of the same reaction they presumed that DAG also increased correspondingly. The effect of increases in IP3 and DAG on membrane potential and excitability, and its relevance to MPR™, has previously been discussed. See U.S. Pat. No. 7,906,300.

Recent clinical trials using human whole blood samples have shown that that the Membrane Potential Ratios (MPR™) are significantly different among Bipolar Disorder (BD) patients. Attention Deficit Hyperactive Disorder (ADHD) patients and the negative group who are neither BD nor ADHD. These experiments involve a test buffer with no K+ ions, but it contains ethyl alcohol (EtOH). The membrane potentials are measured in the test buffer with ethyl alcohol and compared with the membrane potentials measured in a reference buffer without ethyl alcohol. The ratio of the membrane potential (MP) in the test buffer divided by the MP in the reference buffer is called the membrane potential ratio (MPR™). MPR™ values are significantly different in the three groups (see U.S. application Ser. No. 14/236,787, the disclosure of which is incorporated herein by reference in its entirety).

It is generally well recognized that the mental disorders are caused by the malfunction of the neurons in the brain. Neurons communicate with each other through electro-biological signals. These signals are generated and modulated by the membrane potential (MP) and the excitability of the neurons. It is essential to understand the biological basis for these differences in blood cells in order to establish the relationship of these results to neurons and to elucidate the pathophysiology of these illnesses. It is the objective of this effort to discover the common biological pathway that gives rise to the observed differences.

The identification of the molecules that modulate the signaling pathways in the neuronal cell is essential in diagnosing and treating mental illness. The membrane potential is the electrical potential difference (voltage) across a cell's membrane. Membrane potential results from the action of K⁺ ion channels present in the membrane which along with the Na, K-ATPase enzyme maintain viable ion concentrations inside the cell.

Unlike most cells, neurons are electrically active and use changes in membrane potential for fast communication with other neurons. Neurons process and transmit information in the form of electrical signals. K⁺ ion channels in the neuronal membrane set the membrane potentials and the excitability. These signals are then processed, amplified and transmitted to the synapse releasing the neurotransmitters. These transmitters again send a signal through their specific g-protein coupled receptors (GPCR) in the membrane of the target neuron. The GPCRs transmit these signals through two primary signal transduction pathways that process and transmit this signal to the K⁺ ion channels in its membrane. These two pathways are the cAMP signaling pathway and the DAG signaling pathway (Nahorski S. R. British Journal of Pharmacology (2006) 147, S38-S45, the disclosure of which is incorporated herein by reference in its entirety).

Calculations of the membrane potentials (MP) using Goldman-Hodgkin-Katz equation showed that lithium would depolarize the membrane potentials (Thiruvengadam. A. Journal of Affective Disorders 65 (2001) 95-99, the disclosure of which is incorporated herein by reference in its entirety). This result led to the hypothesis that lithium's therapeutic efficacy was due to this depolarizing effect. This result was supported by earlier experimental and clinical results (Yonemura, K. and Sato, M, The Japanese Journal of Physiology, 1967; 17: 678-97; Grafe, et al. Brain Research, 1983; 279: 65-76 and El-Mallakh, et al. J. Affective Disorders, 1996; 41: 33-3; the disclosures of which are incorporated herein by reference in their entirety). Thiruvengadam (Focus on Bipolar Disorder Research ISBN 1-59454-059-4 Editor: Malcomb R. Brown, pp. 15-35, 2005 Nova Science Publishers, Inc.; the disclosure of which is incorporated herein by reference in its entirety) further showed that lithium not only depolarizes the MP but also reduced the excitabilities of neurons. Measurement of membrane potentials of cultured lymphoblasts collected from BD patients showed that the MP was hyperpolarized confirming the measurements of El Mallakh et al. In order to use the MP as a diagnostic marker for BD, a ratiometric method was developed and used successfully for diagnosing BD patients (U.S. Pat. No. 7,425,410B2; the disclosure of which is incorporated herein by reference in its entirety) using their red blood cells (RBC). This Method involves the measurement of MP in two buffers and taking the ratio of these two MPs. These experiments involve a test buffer that contains no K+ ions but contains ethyl alcohol (EtOH). The membrane potentials are measured in the test buffer and compared with the membrane potentials measured in a reference buffer without EtOH. This ratio is called the Membrane Potential Ratio (MPR™). It was further discovered that the MPR™ could also be used to diagnose the ADHD patients (U.S. Pat. No. 7,906,300B2; the disclosure of which is incorporated herein by reference in its entirety).

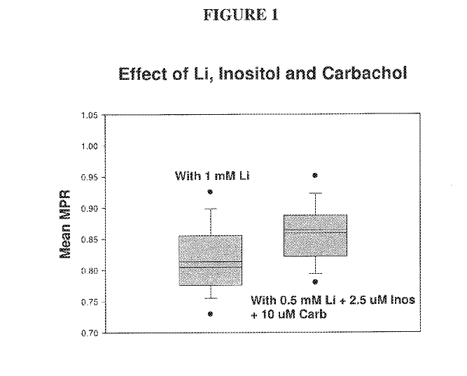

To date, more than 550 patients have been tested using the MPR™. A summary of these test results is shown in FIG. 1. The MPR™ values for BD patients were significantly lower than that for Negatives (including normals, unipolar depressives, and schizophrenics); on the other hand, the MPR™ values for ADHD patients were significantly higher than that for Negatives as shown in FIG. 1.

It is essential to understand the biological basis for these differences in order to establish the scientific mechanisms and the pathways responsible for the differences in the MPR™ among the three groups and to elucidate the pathophysiology of these illnesses.

These signaling pathways and polypeptides can then be used for diagnostic and therapeutic purposes. For example, this invention traces the pathway for BD and ADHD from the G-protein Controlled Receptors (GPCR) to the K⁺ channel in patients' cell. As described in U.S. application Ser. No. 14/236,787, this discovery provided a better understanding of the pathophysiology of these disorders.

CAK Channels and Membrane Potentials in RBC:

Although the expression of one of the small conductance family of CAK channels in RBC has been known since 2003 (Hoffman et al, PNAS 2003 vol. 100 no. 12: 7366-7371), there is no prior art of measuring the MP in RBC leave alone observing the differences among the three groups of patient populations (Negatives, BD and ADHD). Those skilled in the art recognize that the observation that EtOH hyperpolarizes the membrane potentials is a new discovery. Only the experiments using channel blockers, quinine and clotrimazole in RBC established this fact. Patent search as well as literature search using the key words CAK channels and EtOH did not yield any results. CAK channels and MP also did not yield any patents. Adelman et al patent (hSK₂ Channels Adelman et al U.S. Pat. No. 6,797,486) is concerned about hSK₂ DNA sequence and its effect on K⁺ flow throw the channel. Gene sequencing of the hSK4 genes from blood samples drawn from patients did not yield any mutations in the DNA sequence which could explain the MPR™ differences (unpublished results on file).

Ca²⁺/CaM Activation of CAK Channels, EtOH and Membrane Potentials in RBC:

CAK channels are activated by Ca²⁺/CaM is well known in the literature. But it is not obvious from the literature that the membrane potentials can be modulated by either EtOH or by a CaM activator such as CaM Kinase II. A patent search using CaM Kinase II and membrane potentials did not yield any results.

PKC, CaM and Membrane Potentials:

It is well known that PKC through the DAG signaling pathway activates the CaM. However there is no literature indicating that DAG signaling pathway modulates the CaK channels and MP. Those skilled in the art recognize that this is an important discovery.

DAG, CAK Channels and MP:

It is not at all known in the published literature that the DAG has any effect on membrane potentials leave alone in BD and ADHD. There are no patents connecting DAG, MP, BD and ADHD. Caricasole, et al. (DGK Beta Pat. No. 6,593,121 2003) do not address the MPR™ differences and the DAG Pathway that modulates the MPR™. A genome-wide association study implicated the diacylglycerol kinase eta (DGKH) and several other genes in the etiology of bipolar disorder (Baum et al, Mol Psychiatry. 2008 February; 13(2): 197-207). While this study supports this invention it does not a priori recognize the MPR™ as the connecting link via the DAG signaling pathway.

The present methods provide for an increase in the therapeutic efficacy of lithium. In particular, the present invention unexpectedly found that, an increase in the therapeutic efficacy of lithium could be achieved in a combination therapy. The combination therapy allows for a reduction in the dose required to achieve a therapeutic effect for lithium, and this reduces, ameliorates or prevents the side effects associated with lithium treatment.

A combination therapy of the present invention includes a lithium compound and an adjunctive agent.

The adjunctive agent may include, but is not limited to, a cholinergic agent, an immunomodulatory agent, a mood stabilizer agent, an antidepressant agent, an anticonvulsant agent, an antipsychotic agent, and an anxiolytic agent.

A cholinergic agent may include, but is not limited to, a direct cholinergicagonist that binds selectively or non-selectively to a muscarinic or nicotinic receptor and an indirect cholinergic agonist.

An indirect cholinergic agonist may include, but is not limited to, an acetylcholinesterase inhibitor and aM2 receptor antagonist. An acetylcholinesterase inhibitor may include, but is not limited to, donezpezil, galantamine, rivastigmine, tacrine, donepezil/memantine, and pharmaceutically acceptable salts thereof. A M2 receptor antagonist may include, but is not limited to, methoctramine, AF-DX384, and pharmaceutically acceptable salts thereof, an agent that increases the presence of acetylcholine at a muscarinic or nicotinic receptor.

A direct cholinergic agonist that binds selectively or non-selectively to a M1 to M5 muscarinic receptor may include, but is not limited to, acetylcholine, methacholine, arecoline, bethanechol, carbachol, pilocarpine, muscarine, cevimeline, nicotine, and pharmaceutically acceptable salts thereof.

An immunomodulatory agent may include, but is not limited to, levamsiole and pharmaceutically acceptable salts thereof.

A mood stabilizer agent, may include, but is not limited to, valproate, divalproex, carbamazepine, lamotrigine, oxacarabazepine, and pharmaceutically acceptable salts thereof.

An anticonvulsant agent, may include, but is not limited to, lamotrigine, perampanel, mephobarbital, primidone, phenobarbital, diazepam, clonazepam, lorazepam, clobazam, felbamate, topiramate, acetazolamide, zonisamide, rufinamide, oxcarbazepine, carbamazepine, eslicarbazepine, valproic acid, divalproex sodium, gabapentin, gabapentin enacarbil, tiagabine, phenytoin, fosphenytoin, mephenytoin, ethotoin, magnesium sulfate, lacosamide, ezogabine, trimethadione, levetiracetam, ethosuximide, methsuximide, and pharmaceutically acceptable salts thereof.

An antidepressant agent may include, but is not limited to, fluoxetine, ariprazole, doxepin, clomipramine, bupropion, amoxapine, nortriptyline, vortioxetine, citalopram, duloxetine, trazodone, venlafaxine, selegiline, perphenazine, amitriptyline, levomilnacipram, desvenlafaxine, lurasidone, lamotrigine, escitalopram, chlordiazepoxide, isocarboxazid, phenelzine, desipramine, trazodone, tranylcypromine, paroxetine, mirtazapine, quetiapine, nefazodone, doxepin, trimipramine, imipramine, vilazodone, protriptyline, sertraline, olanzapine, and pharmaceutically acceptable salts thereof.

An anxiolytic agent may include, but is not limited to, secobarbital, mephobarbital, pentobarbital, phenobarbital, amobarbital, butabarbital, estazolam, alprazolam, flurazepam, diazepam, chlordiazepoxide, clorazepate, clonazepam, oxazepam, diazepam, triazolam, lorazepam, temazepam, midazolam, clobazam, diphenhydramine, zolpidem, chloral hydrate, doxepin, sodium oxybate, doxylamine, doxepin, hydroxyzine, meprobamate, ethchlorvynol, eszopiclone, buspirone, zalephon, ramelteon, suvorexant, tryptophan, tasimelteon, dexmedetomidine, and pharmaceutically acceptable salts thereof.

An antipsychotic agent, may include, but is not limited to, haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof.

In some embodiments, the cholinergic agonist may be, for example, one or more of acetylcholine, nicotine, muscarine, carbachol, galantamine, arecoline, cevimeline, levamisole, clozapine and donepezil.

As used herein, “an effective amount,” “a therapeutically effective amount” or “an effective dosage” is one which reduces symptoms of the BD condition or pathology, and preferably which normalizes physiological responses in an individual with the BD condition or pathology. MPR™ may be used to identify the “effective amount,” the therapeutically effective amount” or the “effective dosage” directly through a blood test. For instance, the effective amount of an amount of lithium and/or the effective amount of an adjunctive agent is an amount which brings the diagnostic probability to the negative range as discussed U.S. application Ser. No. 14/236,787, the disclosure of which is incorporated herein in its entirety. As exemplified in Example 4 below, in a BD patient, the MPR™ returns to negative with treatment using an effective amount. This an example of how an “effective amount” or “effective dosage” can be determined.

Reduction of symptoms or normalization of physiological responses can be determined using methods routine in the art for assessing BD. In one aspect, “an effective amount” or a “therapeutically effective amount” of a lithium compound and/or “an effective amount” or a “therapeutically effective amount” of at least one adjunctive agent of the invention, or a pharmaceutical combination or composition comprising the same of the invention, is an amount which restores a measurable physiological parameter, such as the membrane potential, to substantially the same value (for instance, preferably to within 30% or less, more preferably to within 20% or less, and still more preferably, to within 10% or less) of the value of the parameter in an individual without BD disease condition or pathology. In another aspect. “an effective amount” or a “therapeutically effective amount” of a lithium compound and/or “an effective amount” or a “therapeutically effective amount” of at least one adjunctive agent of the invention, or a pharmaceutical combination or composition comprising the same of the invention, is an amount which restores a measurable physiological parameter, such as the membrane potential, to a value substantially higher than (preferably at least 10% higher than, more preferably at least 20% higher than, and still more preferably at least 30% higher than) the parameter of a BD control individual. The percentage may be determined by a clinician treating the patient. The criteria may be whether the effective amount brings down the diagnostic probability to the negative range. The dosage may be adjusted or vary according to the patient response to lithium and/or an adjunctive agent, or the patient response to the synergistic combination.

In one embodiment, an “effective amount” or “therapeutically effective amount” may be associated with an amount sufficient to provide a therapeutically efficacious plasma level of a drug, as may be determined during clinical treatment. A “therapeutically efficacious plasma level” is the amount of the drug (such as a lithium compound or an adjunctive agent) present in the blood sufficient to produce a therapeutic effect.

For instance, an “effective amount” or “therapeutically effective amount” may be associated with an amount sufficient to provide a plasma lithium level of 2.0 mM or less, preferably a plasma lithium level of 1.2 mM or less, preferably a plasma lithium level of 1 mM or less, a plasma lithium level of from 0.5 mM to 1.2 mM, a plasma lithium level of from 0.8 mM to 1.2 mM, more preferably, a plasma lithium level of from 0.6 mM to 0.75 mM, or more preferably a plasma lithium level of from 0.4 mM to 0.6 mM. More preferably, an “effective amount” or “therapeutically effective amount” of a lithium compound, may be associated with an amount sufficient to provide a plasma lithium level of at least 1 mM, a plasma lithium level of at least 0.8 mM, preferably, a plasma lithium level of at least 0.5 mM, or a plasma lithium level of at least 0.4 mM. This effective amount or therapeutically effective amount may be determined clinically, and the amount of lithium or adjunctive agent sufficient to provide the above plasma lithium levels may be an amount less than that used in current BD drug therapy, since certain drugs described herein may increase the DAG concentration by 10 fold.

In another embodiment, an “effective amount” or “therapeutically effective amount” may be associated with an amount sufficient to provide a plasma lithium level of 2.0 mEq/L or less, preferably a plasma lithium level of 1.2 mEq/L or less, a plasma lithium level of 1 mEq/L or less, a plasma lithium level of from 0.5 mEq/L to 1.2 mEq/L, a plasma lithium level of from 0.8 mEq/L to 1.2 mEq/L, more preferably, a plasma lithium level of from 0.6 mEq/L to 0.75 mEq/L, or more preferably a plasma lithium level of from 0.4 mEq/L to 0.6 mEq/L. More preferably, an “effective amount” or “therapeutically effective amount” of a lithium compound, may be associated with an amount sufficient to provide a plasma lithium level of at least 1 mEq/L, a plasma lithium level of at least 0.8 mEq/L, preferably, a plasma lithium level of at least 0.5 mEq/L, or a plasma lithium level of at least 0.4 mEq/L. This amount may be determined clinically, and may depend on the adjunctive drug used with lithium in a drug combination, so that the effective amount may be determined to be associated with a plasma lithium level as low as 0.1 mEq/L (up to 1.2 mEq/L). Preferably, the effective amount of lithium in the drug combination of the present invention, is an amount less than that used in current BD drug therapy.

Likewise, as is apparent to one skilled in the art, an “effective amount” or “therapeutically effective amount” of an adjunctive agent described herein may be associated with an amount sufficient to provide a therapeutically efficacious plasma level of the respective adjunctive agent. This amount may also be determined through clinical treatment. The “effective amount” or “therapeutically effective amount” amount of an adjunctive agent maybe determined based on a plasma lithium level as described above. Preferably, the effective amount of an adjunctive agent in the drug combination of the present invention, is an amount less than that used in its current drug therapy.

The “effective amount,” “therapeutically effective amount” or the “effective dosage” may be an amount of lithium that is sufficient to interact synergistically with at least one adjunctive agent, to improve or enhance the therapeutic effect or therapeutically efficacious plasma level of the adjunctive agent; and/or an amount of at least one adjunctive agent that is sufficient to interact synergistically with lithium to improve or enhance the therapeutic effect or therapeutically efficacious plasma level of lithium.

Non-limiting examples of therapeutically efficacious plasma levels of adjunctive agents useful in the present invention are exemplified below.

Amitriptyline: 120 to 150 ng/mL

Carbamazepine: 5 to 12 μg/mL
Nortriptyline: 50 to 150 ng/mL
Phenobarbital: 10 to 30 μg/mL
Phenytoin: 10 to 20 μg/mL
Valproic acid: 50 to 100 μg/mL

Preferably, the effective amount of lithium compound is a dose amount that is less than a dosage of lithium required to provide a therapeutic effect in current BD therapy when used alone, or is a dose amount that is less than a dosage of lithium required in current BD therapy when used alone to provide a therapeutically efficacious plasma lithium level for BD therapy. For instance, the effective dose may be a dose that brings the diagnostic probability to the negative range. Likewise, the effective amount of at least one adjunctive agent may include a dose that is less than a dosage of the at least one adjunctive agent required to provide a therapeutically efficacious plasma level of the at least one adjunctive agent when administered alone.

As is apparent to one skilled in the art, an “effective amount” or a “therapeutically effective amount” of a lithium compound and/or “an effective amount” or a “therapeutically effective amount” of at least one adjunctive agent of the invention, or a pharmaceutical combination or composition comprising the same of the present invention, will also vary depending upon the age, weight and mammalian species treated, the particular compounds employed, the particular mode of administration and the desired effects and the therapeutic indication. Because these factors and their relationship to determining this amount are well known, the determination of an effective dosage level or therapeutically effective dosage levels—such as the amount necessary to achieve the desired result therapeutically efficacious plasma level of lithium or therapeutically efficacious plasma level of an adjunctive agent described herein—will be within the skill of the skilled person. Alternatively, the determination of an effective dosage level or therapeutically effective dosage levels—the amount which restores a measurable physiological parameter such as the membrane potential to substantially the same value to the negative range as exemplified in Example 4 (preferably to within 30% or less, more preferably to within 20% or less, and still more preferably, to within 10% or less) of the value of the parameter in an individual without BD disease condition or pathology, or the amount which restores a measurable physiological parameter, such as the membrane potential, to a value substantially higher than (preferably at least 10% higher than, more preferably at least 20% higher than, and still more preferably at least 30% higher than) the parameter of a BD control individual—will be within the skill of the skilled person.

For instance, an “effective amount” or a “therapeutically effective amount” of a lithium compound or of at least one adjunctive agent of the present invention, or a pharmaceutical combination or composition of the present invention, will depend on the route of administration, the type of mammal being treated, and the physical characteristics of the specific mammal under consideration. These factors and their relationship to determining this amount are well known to skilled practitioners in the medical arts. This amount and the method of administration can be tailored to achieve optimal efficacy so as to deliver the agent, pharmaceutical combination, or pharmaceutical composition to the BD patient, but will depend on such factors as weight, diet, concurrent medication and other factors, well known to those skilled in the medical arts.

In some combination therapies of the present invention, the combination or composition comprising lithium and the at least one cholinergic agonist may be present together in a single dosage form, or may be present in separate dosage forms. For different patients, and even for the same patient over time (for example, if the symptoms of bipolar disorder improve or worsen; or for example, depending on the result of a mean membrane potential test from cells obtained from a BD patient during or after therapy), the dosage of lithium and/or the at least one cholinergic agonist may be increased or decreased. The process of adjusting dosages in an upward or downward direction and evaluating the effect of the adjustment on mean membrane potential, and/or BD symptoms, may be continued until an optimum dosage is determined to bring the diagnostic probability to the negative range at which the patient experiences the best balance between therapeutic effectiveness and side-effects.

Dosages of the lithium compound and at least one adjunctive agent (such as a cholinergic agonist) may vary depending on such factors as, for example, the characteristics of the patient, and the frequency of administration.

The at least one adjunctive agent (such as a cholinergic agonist) may be administered such that the patient is provided with a therapeutically-effective plasma concentration thereof. In some embodiments, where the cholinergic agonist is carbachol, the patient may be provided with a plasma concentration of 30 μM or less, 25 μM or less, 20 μM or less, 15 μM or less, 10 μM or less, 9 μM or less, 8 μM or less, 7 μM or less, 6 μM or less, 5 μM or less, 4 μM or less, 3 μM or less, or 2 μM or less. Alternatively, the optimum concentration may be determined based on the patient's individual factors or may be determined through patient clinical trials using the diagnostic probability as the criterion as described earlier.

In some embodiments, where the cholinergic agonist is clozapine, the patient is provided with a plasma concentration of 500 ng/ml or less, 400 ng/ml or less, 300 ng/ml or less, 200 ng/ml or less, 150 ng/ml or less, 100 ng/ml or less, 90 ng/ml or less, 80 ng/ml or less, 70 ng/ml or less, 60 ng/ml or less, 50 ng/ml or less, 40 ng/ml or less, 30 ng/ml or less, 20 ng/ml or less, or 10 ng/ml or less. Alternatively, the optimum concentration may be determined based on the patient's individual factors or may be determined through patient clinical trials using the diagnostic probability as the criterion as described earlier.

In some embodiments, where the cholinergic agonist is donepezil, the patient is provided with a plasma concentration of 50 ng/ml or less, 40 ng/ml or less, 30 ng/ml or less, 20 ng/ml or less, 10 ng/ml or less, 9 ng/ml or less, 8 ng/ml or less, 7 ng/ml or less, 6 ng/ml or less, 5 ng/ml or less, 4 ng/ml or less, 3 ng/ml or less, or 2 ng/ml or less. Alternatively, the optimum concentration may be determined based on the patient's individual factors or may be determined through patient clinical trials using the diagnostic probability as the criterion as described earlier.

The biochemical form of lithium is not strictly limited. In some embodiments, the lithium may be in the form of lithium carbonate. However, other salt forms that could serve as a source of lithium include, for example: lithium benzoate, lithium bromide, lithium cacodylate, lithium caffeine sulfonate, lithium chloride, lithium citrate, lithium dithiosalicylate, lithium formate, lithium glycerophosphate, lithium iodate and lithium salicylate. The lithium salts may be given in a substantially pure form or mixed with other compounds, foods, or therapeutic agents as the exigencies of individual cases require.

The lithium and/or the at least one adjunctive agent (such as a cholinergic agonist) of the combination therapy of the present invention may be administered separately or together, with or without a pharmaceutically acceptable carrier or vehicle. They can be provided in dosage forms such as tablets, capsules, powder packets, or liquid solutions for oral administration. Methods for preparing these dosage forms are well known in the art (see, e.g., Remington's Pharmaceutical Sciences, 16th Ed., A. Oslo Ed. Mack. Easton. Pa. (1980), incorporated herein by reference in its entirety). When given orally, therapeutically inert agents may be added to improve palatability, or additional therapeutic agents may be added. Pharmaceutically acceptable carriers include diluents and excipients generally used in pharmaceutical preparations, such as fillers, extenders, binders, moisturizers, disintegrators, surfactants, and lubricants. The lithium and/or the at least one cholinergic agonist of the combination therapy of the present invention may be formulated as a pharmaceutical preparation, for example in the form of tablets, flash melt tablets, pills, powder, liquid, suspension, emulsion, granules, capsules, suppositories or injection (liquid, suspension, etc.), troches, intranasal spray percutaneous patch and the like.

In case of shaping to tablet formulation, a wide variety of carriers that are known in this field can be used. Examples include lactose, saccharose, sodium chloride, glucose, urea, starch, xylitol, mannitol, erythritol, sorbitol, calcium carbonate, kaolin, crystalline cellulose, silic acid and other excipients; water, ethanol, propanol, simple syrup, glucose solution, starch solution, gelatin solution, carboxymethyl cellulose, shellac, methyl cellulose, potassium phosphate, polyvinyl pyrrolidone and other binders; dried starch, sodium alginate, agar powder, laminaran powder, sodium hydrogencarbonate, calcium carbonate, polyoxyethylenesorbitan fatty acid esters, sodium lauryl sulfate, stearic acid monoglyceride, starch, lactose and other disintegrators; white sugar, stearin, cacao butter, hydrogenated oil and other disintegration inhibitors; quaternary ammonium salt, sodium lauryl sulfate and other absorption accelerator; glycerine, starch and other moisture retainers; starch, lactose, kaolin, bentonite, colloidal silic acid and other adsorbents; and refined talc, stearate, boric acid powder, polyethylene glycol and other lubricants and the like. Tablets can also be formulated if necessary as tablets with ordinary coatings, such as sugar-coated tablets, gelatin-coated tablets, enteric coated tablets and film coated tablets, as well as double tablets and multilayered tablets.

In case of shaping to pills, a wide variety of carriers that are known in this field can be used. Examples include glucose, lactose, starch, cacao butter, hardened vegetable oil. kaolin, talc and other excipients; gum arabic powder, traganth powder, gelatin, ethanol and other binders; and laminaran, agar and other disintegrators and the like.

In case of shaping to a suppository formulation, a wide variety of carriers that are known in the field can be used. Examples include polyethylene glycol, cacao butter, higher alcohol, esters of higher alcohol, gelatin semi-synthetic glyceride and the like.

In addition, colorants, preservatives, perfumes, flavorings, sweeteners and the like as well as other drugs may be contained in the pharmaceutical composition.

Individual preparations of a cholinergic agonist and lithium may also be provided in the form of a kit, comprising a carrier (e.g. a box or bag) compartmentalized to receive one or more components (bottles, vials, packets etc.) in close confinement. It is expected that such a kit would be carried by patients with bipolar disorder and that it would contain written instructions concerning the way in which the enclosed drugs should be taken, potential side effects, etc. The kit should be portable, and be generally convenient for use by patients.

For parenteral administration, preparations containing lithium and/or at least one cholinergic agonist may be provided to patients in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. Intravenous vehicles include fluid and nutrient replenishers, electrolyte replenishers, such as those based upon Ringer's dextrose and the like.

The methods for administration of the pharmaceutical composition of the present invention are not specifically restricted. The composition is administered depending on each type of preparation form, and the age, gender and other condition of the patient (degree and conditions of the disease, etc.). For example, tablets, pills, liquids, suspensions, emulsions, granules and capsules are administered orally. In case of injection preparation, it is administered intravenously either singly or mixed with a common auxiliary liquid such as solutions of glucose or amino acid. Further, if necessary, the injection preparation is singly administered intradermally, subcutaneously or intraperitoneally. In case of a suppository, it is administered intrarectally.

The patient may be administered the combination therapy several times per day, once per day, once every other day, or once per week or less. The lithium compound and at least one adjunctive agent contemplated herein may be administered, simultaneously with or sequentially (such as prior to or after), in combined or separate formulation(s), in a coordinate treatment protocol. In certain embodiments, a lithium compound is administered coordinately with at least one adjunctive agent contemplated herein, using separate formulations or a combinatorial formulation as described herein (i.e., comprising both a lithium compound, and at least one adjunctive agent). This coordinate administration may be done simultaneously or sequentially in either order, and there may be a time period while only one or both (or all) active therapeutic agents individually and/or collectively exert their biological activities.

The combination therapies of the present invention may include, in addition to lithium and at least one adjunctive agent such as, one or more of 1) mood stabilizers such as Cibalith, Eskalith, Lithane, Litho-tabs, and Lithobid; 2) anti-psychotics such as Abilify, Geodon, Haldol, Risperdol, Saphris, Seroquel, Zyprexa, and Symbyax; 3) anti-anxiety Drugs such as Ativan, Klonopin, Valium, and Xanax; and/or 4) anti-convulsants such as Depakote, Lamictal, and Tegretol.

In the present invention, one method for determining the optimum dose of a combination therapy for the treatment of BD, or for monitoring the efficacy of a combination therapy for the treatment of BD, is to determine the membrane potential ratio (MPR™) of cells obtained from the BD patient. The MPR™ test has been described in U.S. Pat. Nos. 7,425,410 and 7,906,300, as well as U.S. Provisional Application Nos. 61/543,061 and 61/653,579, which are hereby incorporated by reference in their entirety. Briefly, the MPR™ test involves measuring the membrane potential of the human cells in a test buffer and in a reference buffer, and calculating the ratio of these membrane potentials. U.S. Pat. Nos. 7,425,410 and 7,906,300 describe the use of this method to diagnose BD; however, it can also be used to determine the optimum dose of a combination therapy for the treatment of BD, or to monitor the efficacy of a combination therapy for the treatment of BD, by measuring and/or adjusting the MPR™ values. For example, in some embodiments, if the BD patients respond to the combination therapy then the MPR™ values return to the negative range. Otherwise the treatment protocol is adjusted appropriately till the MPR™ values reach the negative range.

The membrane potentials of whole blood cells can be measured using two different buffers in a plate reader. The mean MPR™ value is the ratio between the membrane potential of a patient's cells in the test buffer as the numerator and that in the reference buffer as the denominator (for example, determined by statistical analysis of multiple measurements, using the ANOVA and the multiple statistical regression analysis). See Thiruvengadam et al., J Affect Disord 100(1-3):75-82 (2007), which is hereby incorporated by reference in its entirety.

In some aspects, the present invention relates to determining the optimum dose of a combination therapy for the treatment of BD, by analyzing the membrane potential of cells isolated from a BD patient treated with the combination therapy, and calculating a membrane potential ratio therefrom.

### First Embodiment

In one embodiment, a method of determining an optimal combination drug treatment therapy for a patient with bipolar disorder (BD), is provided that comprises:

obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K⁺ or absence of K⁺ (preferably, the test buffer is in the absence of K⁺ (i.e., both reference buffer and test buffer do not have K⁺);

comparing the ratio of the mean membrane potential to (a) and/or (b):
(a) a control ratio of a mean membrane potential of first population of control human cells known to not have BD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+.
(b) a bipolar control ratio of a mean membrane potential of first population of bipolar control human cells known to have BD incubated in vitro in the presence of the agent that alters diacylglycerol signaling and in the absence of K+, to a mean membrane potential of a second population of the bipolar control human cells incubated in vitro in the absence of the agent that alters diacylglycerol signaling and in the presence of K+ or absence of K+.

identifying the optimal combination drug treatment therapy when the ratio of the mean membrane potential obtained is not significantly different from the control ratio of (a), is increased towards the control ratio (a) in comparison to or relative to the bipolar control ratio of (b), and/or is significantly higher in comparison to or relative to the BD control ratio of (b).

The method may further include obtaining an initial ratio of a mean membrane potential from an initial population of cells from the human patient before the obtaining step.

The human cells that may be used in the present method include, but is not limited to, red blood cells, lymphoblasts, crythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a lithium compound and at least one adjunctive agent.

The lithium compound may be present in an effective amount sufficient to provide a plasma lithium level of 1 mM or less, more preferably, the plasma lithium level is 0.5 mM.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range. Preferably, the effective amount of lithium is the dosage amount that improves or enhances the therapeutic effect or therapeutically efficacious plasma level of an adjunctive agent.

The at least one adjunctive agent used in the method may include, but is not limited to, a mood stabilizer, an anticonvulsant, an antipsychotic, an anxiolytic, and a cholinergic agonist, as described herein.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of lithium.

The agent that alters diacylglycerol signaling may include, but is not limited to, a calcium-calmodulin (Ca²⁺/CaM) kinase inhibitor, a diacylglycerol kinase inhibitor, a protein kinase C inhibitor, and an agent that affects calcium-activated potassium (CaK) channels.

Preferably, the agent is a calcium-calmodulin (Ca²⁺/CaM) kinase inhibitor, such as autocamtide-2-related inhibitory peptide (AIP).

Preferably, the agent is a diacylglycerol kinase inhibitor, such as 6-[2-[4-[(4-fluorophenyl)phenylmethylene]-1-piperidinyl]ethyl]-7-methyl-5H-thiazolo[3,2-alpyrimidin-5-one (ALX).

The mean membrane potential test may further include incubating the cells in vitro in buffer comprising a potential-sensitive dye, resuspending the cells in potential-sensitive dye free-buffer, and measuring the cell fluorescence.

The agent that alters K⁺ channel activity may include, but is not limited to, ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

### Second Embodiment

In a second embodiment, the present invention provides a method of optimizing a combination drug treatment therapy for a patient with bipolar disorder (BD), comprising the steps of:

obtaining at least one sample from a BD patient in a drug therapy treatment for BD;

performing on each sample, a mean membrane potential test comprising:
obtaining a ratio of a mean membrane potential that is a mean membrane potential of a first population of cells from the sample incubated in vitro in the presence of an agent that alters diacylglycerol signaling and in the absence of K⁺, to a mean membrane potential of a second population of the sample incubated in vitro in the absence of the test agent that alters diacylglycerol signaling and in the presence of K⁺ or absence of K⁺;
comparing the ratio of the mean membrane potential to (a) and/or (b):
   (a) a control ratio of a mean membrane potential of a first population of control human cells known to not have BD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+,

(b) a bipolar control ratio of a mean membrane potential of a first population of bipolar control human cells known to have BD incubated in vitro in the presence of the agent that alters diacylglycerol signaling and in the absence of K+, to a mean membrane potential of a second population of the bipolar control human cells incubated in vitro in the absence of the agent that alters diacylglycerol signaling and in the presence of K+ or absence of K+;

determining an optimal drug therapy treatment for the BD patient when the ratio of the mean membrane potential obtained is not significantly different from the control ratio of (a), is increased towards the control ratio (a) in comparison to or relative to the bipolar control ratio of (b), and/or is significantly higher in comparison to or relative to the BD control ratio of (b).

The method optionally includes modifying at least one drug in the drug therapy treatment for BD when the least one drug treatment therapy for BD is determined to not be the optimal drug therapy treatment. Such as when the ratio of the mean membrane potential obtained is lower in comparison to or relative to the control ratio of (a), is decreased towards the bipolar control ratio of (b) in comparison to or relative to the control ratio of (a), and/or is not significantly different from the BD control ratio in (b).

The method may further include obtaining an initial ratio of a mean membrane potential from an initial population of cells from the human patient before the obtaining step.

The human cells that may be used in the present method include, but is not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a lithium compound and at least one adjunctive agent.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

The lithium compound may be present in an effective amount sufficient to provide a plasma lithium level of 1 mM or less, more preferably, the plasma lithium level is 0.5 mM.

The at least one adjunctive agent used in the method may include, but is not limited to, a mood stabilizer, an anticonvulsant, an antipsychotic, an anxiolytic, and a cholinergic agonist, as described herein.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of lithium.

The agent that alters diacylglycerol signaling may include, but is not limited to, a calcium-calmodulin (Ca²⁺/CaM) kinase inhibitor, a diacylglycerol kinase inhibitor, a protein kinase C inhibitor, and an agent that affects calcium-activated potassium (CaK) channels.

Preferably, the agent is a calcium-calmodulin (Ca²+/CaM) kinase inhibitor, such as autocamtide-2-related inhibitory peptide (AIP).

Preferably, the agent is a diacylglycerol kinase inhibitor, such as 6-[2-[4-[(4-fluorophenyl)phenylmethylene]-1-piperidinyl]ethyl]-7-methyl-5H-thiazolo[3,2-a]pyrimidin-5-one (ALX).

The mean membrane potential test may further include incubating the cells in vitro in buffer comprising a potential-sensitive dye, resuspending the cells in potential-sensitive dye free-buffer, and measuring the cell fluorescence.

The agent that alters K⁺ channel activity may include, but is not limited to, ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

### Third Embodiment

In a third embodiment, the present invention provides a method for determining an optimum dosage of a drug in a combination drug treatment therapy for the treatment of bipolar disorder (BD), said method comprising:

obtaining at least one sample from a BD patient treated with a dosage of a drug in a combination therapy;

performing on each sample, a mean membrane potential test comprising:
obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K+ or absence of K⁺;
comparing the ratio of the mean membrane potential to (a) and/or (b):
   (a) a control ratio of a mean membrane potential of a first population of cells from a control human known to not have said BD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+.
   (b) a bipolar control ratio of a mean membrane potential of a first population of cells from a bipolar control human known to have said BD incubated in vitro in the presence of the agent that alters diacylglycerol signaling and in the absence of K+, to a mean membrane potential of a second population of cells from the bipolar control human incubated in vitro in the absence of the agent that alters diacylglycerol signaling and in the presence of K+ or absence of K+;

determining the dosage of the drug in the combination drug treatment therapy is an optimal dosage for treating BD in the combination therapy when the ratio of the mean membrane potential obtained is not significantly different from the control ratio of (a), is increased towards the control ratio (a) in comparison to or relative to the bipolar control ratio of (b), and/or is significantly higher in comparison to or relative to the BD control ratio of (b).

The method may further optionally include determining the dosage of the drug in the combination drug treatment therapy is not the optimal dosage for treating BD in the combination therapy when the ratio of the mean membrane potential obtained is lower in comparison to or relative to the control ratio of (a), is decreased towards the bipolar control ratio of (b) in comparison to or relative to the control ratio of (a), and/or is not significantly different from the BD control ratio of (b).

The method may further optionally include modifying the dosage of the drug in the combination drug treatment therapy when the dosage of the drug in the combination therapy is determined to be not the optimal dosage for treating BD based on the mean membrane potential test.

The method may further include obtaining an initial ratio of a mean membrane potential from an initial population of cells from the human patient before the obtaining step.

The human cells that may be used in the present method include, but is not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a lithium compound and at least one adjunctive agent.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

The lithium compound may be present in an effective amount sufficient to provide a plasma lithium level of 1 mM or less, more preferably, the plasma lithium level is 0.5 mM

The at least one adjunctive agent used in the method may include, but is not limited to, a mood stabilizer, an anticonvulsant, an antipsychotic, an anxiolytic, and a cholinergic agonist, as described herein.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of lithium.

The agent that alters diacylglycerol signaling may include, but is not limited to, a calcium-calmodulin (Ca²⁺/CaM) kinase inhibitor, a diacylglycerol kinase inhibitor, a protein kinase C inhibitor, and an agent that affects calcium-activated potassium (CaK) channels.

Preferably, the agent is a calcium-calmodulin (Ca²⁺/CaM) kinase inhibitor, such as autocamtide-2-related inhibitory peptide (AIP).

Preferably, the agent is a diacylglycerol kinase inhibitor, such as 6-[2-[4-[(4-fluorophenyl)phenylmethylene]-1-piperidinyl]ethyl]-7-methyl-5H-thiazolo[3,2-a]pyrimidin-5-one (ALX).

The mean membrane potential test may further include incubating the cells in vitro in buffer comprising a potential-sensitive dye, resuspending the cells in potential-sensitive dye free-buffer, and measuring the cell fluorescence.

The agent that alters K⁺ channel activity may include, but is not limited to, ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

### Fourth Embodiment

In a fourth embodiment, the present invention provides a method for monitoring the efficacy of a combination drug treatment therapy for the treatment of bipolar disorder (BD), said method comprising:

obtaining at least one sample from a BD patient treated with a combination drug treatment therapy for treating BD;

performing on each sample, a mean membrane potential test comprising:
obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K⁺ or absence of K+;
comparing the ratio of the mean membrane potential to (a) and/or (b):
   (a) a control ratio of a mean membrane potential of a first population of cells from a control human known to not have said BD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+.
   (b) a bipolar control ratio of a mean membrane potential of a first population of cells from a bipolar control human known to have said BD incubated in vitro in the presence of the agent that alters diacylglycerol signaling and in the absence of K+, to a mean membrane potential of a second population of cells from the bipolar control human incubated in vitro in the absence of the agent that alters diacylglycerol signaling and in the presence of K+ or absence of K+;

determining the combination drug treatment therapy is efficacious based on the mean membrane potential test when the ratio of the mean membrane potential obtained is not significantly different from the control ratio of (a), is increased towards the control ratio in comparison to or relative to the bipolar control ratio of (b), and/or is significantly higher in comparison to or relative to the BD control ratio of (b)

The method may optionally further include determining the combination drug treatment therapy is not efficacious based on the mean membrane potential test when the ratio of the mean membrane potential obtained is lower in comparison to or relative to the control ratio of (a), is decreased towards the bipolar control ratio of (b) in comparison to or relative to the control ratio of (a), and/or is not significantly different from the BD control ratio of (b).

The method may optionally further include adjusting a dosage of one or more agents in the combination drug treatment therapy when the combination therapy is determined to be not efficacious based on the mean membrane potential test.

The method may further include obtaining an initial ratio of a mean membrane potential from an initial population of cells from the human patient before the obtaining step.

The human cells that may be used in the present method include, but is not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a lithium compound and at least one adjunctive agent.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

The lithium compound may be present in an effective amount sufficient to provide a plasma lithium level of 1 mM or less, more preferably, the plasma lithium level is 0.5 mM.

The at least one adjunctive agent used in the method may include, but is not limited to, a mood stabilizer, an anticonvulsant, an antipsychotic, an anxiolytic, and a cholinergic agonist, as described herein.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of lithium.

The agent that alters diacylglycerol signaling may include, but is not limited to, a calcium-calmodulin (Ca²⁺/CaM) kinase inhibitor, a diacylglycerol kinase inhibitor, a protein kinase C inhibitor, and an agent that affects calcium-activated potassium (CaK) channels.

Preferably, the agent is a calcium-calmodulin (Ca²⁺/CaM) kinase inhibitor, such as autocamtide-2-related inhibitory peptide (AIP).

Preferably, the agent is a diacylglycerol kinase inhibitor, such as 6-[2-[4-[(4-fluorophenyl)phenylmethylene]-1-piperidinyl]ethyl]-7-methyl-5H-thiazolo[3,2-a]pyrimidin-5-one (ALX).

The mean membrane potential test may further include incubating the cells in vitro in buffer comprising a potential-sensitive dye, resuspending the cells in potential-sensitive dye free-buffer, and measuring the cell fluorescence.

The agent that alters K⁺ channel activity may include, but is not limited to, ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof. Preferably, the agent that alters K⁺ channel activity is ethanol.

### Fifth Embodiment

In a fifth embodiment, the present invention provides a method of treating bipolar disorder (BD), comprising administering an effective amount of a lithium compound and at least one adjunctive agent to a human patient with BD.

The at least one adjunctive agent and the lithium compound may form a synergistic combination or composition to treat BD.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

The effective amount of lithium compound may be a dose amount that is less than a dosage of lithium required to provide a therapeutically efficacious plasma lithium level for BD therapy when used alone. Preferably, the dose provides a plasma lithium level of 1 mM or less. More preferably, the dose provides a plasma lithium level of 0.5 mM or less.

The at least one adjunctive agent may be administered at a dose that is less than a dosage of the at least one adjunctive agent required to provide a therapeutically efficacious plasma level of the at least one adjunctive agent when administered alone.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of lithium.

Preferably, the at least one adjunctive agent is a cholinergic agonist including, but not limited to, donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof. Preferably, the cholinergic agonist is carbachol and pharmaceutically acceptable salts thereof, or donepezil and pharmaceutically acceptable salts thereof. Carbachol may be administered to provide a plasma concentration of 10 μM or less. Donepezil may be administered to provide a plasma concentration of 10 ng/ml or less.

Preferably, the antipsychotic may include, but is not limited to, haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof. Preferably, the antipsychotic is clozapine and pharmaceutically acceptable salts thereof. Clozapine may be administered to provide a plasma concentration of 100 ng/ml or less.

### Sixth Embodiment

In a sixth embodiment, the present invention provides a method of increasing the therapeutic efficacy of a lithium compound for the treatment of bipolar disorder (BD), comprising administering an effective amount of a lithium compound with at least one adjunctive agent, to a human patient with BD.

The at least one adjunctive agent and the lithium compound may form a synergistic combination or composition to treat BD.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

The effective amount of lithium compound may be a dose amount that is less than a dosage of lithium required to provide a therapeutically efficacious plasma lithium level for BD therapy when used alone. Preferably, the dose provides a plasma lithium level of 1 mM or less. More preferably, the dose provides a plasma lithium level of 0.5 mM or less.

The at least one adjunctive agent may be administered at a dose that is less than a dosage of the at least one adjunctive agent required to provide a therapeutically efficacious plasma level of the at least one adjunctive agent when administered alone.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of lithium.

Preferably, the at least one adjunctive agent is a cholinergic agonist including, but not limited to, donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof. Preferably, the cholinergic agonist is carbachol and pharmaceutically acceptable salts thereof, or donepezil and pharmaceutically acceptable salts thereof. Carbachol may be administered to provide a plasma concentration of 10 μM or less. Donepezil may be administered to provide a plasma concentration of 10 ng/ml or less.

Preferably, the antipsychotic may include, but is not limited to, haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof. Preferably, the antipsychotic is clozapine and pharmaceutically acceptable salts thereof. Clozapine may be administered to provide a plasma concentration of 100 ng/ml or less.

### Seventh Embodiment

The invention further provides a method of determining an optimal combination drug treatment therapy for a patient with BD, that comprises:

obtaining a ratio of a mean membrane potential that is a mean membrane potential of a first population of cells from the BD patient incubated in vitro in the presence of an agent that alters human calcium-activated potassium channels (hSK₄) activity and in the absence of K⁺, to a mean membrane potential of a second population of the human patient cells incubated in vitro in the absence of the test agent that alters human calcium-activated potassium channels (hSK₄) activity and the presence of K⁺ or absence of K⁺;

comparing the test ratio to (a) and/or (b):

(a) a control ratio of a mean membrane potential of control human cells known to not have said BD incubated in vitro in the presence of the agent that alters human calcium-activated potassium channels hSK₄ and in the absence of K⁺, to a mean membrane potential of the control human cells incubated in vitro in the absence of the agent that alters human calcium-activated potassium channels hSK₄ and in the presence of K⁺ or absence of K⁺,

(b) a bipolar control ratio of a mean membrane potential of bipolar control human cells known to have said BD incubated in vitro in the presence of the agent that alters human calcium-activated potassium channels hSK₄ and in the absence of K⁺, to a mean membrane potential of the bipolar control human cells incubated in vitro in the absence of the agent that alters human calcium-activated potassium channels hSK₄ and in the presence of K⁺ or absence of K⁺;

identifying the optimal combination drug treatment therapy when the ratio of the mean membrane potential is not significantly different from the control ratio of (a), is increased towards the control ratio (a) in comparison to or relative to the bipolar control ratio of (b), and/or is significantly higher in comparison to or relative to the bipolar ratio of (b).

The method may further include obtaining an initial ratio of a mean membrane potential from an initial population of cells from the human patient before the obtaining step.

The agent that may be used include, but is not limited to, a calcium-calmodulin (Ca²⁺/CaM) kinase inhibitor, a diacylglycerol kinase inhibitor, and a PKC inhibitor. Preferably, the agent is a calcium-calmodulin (Ca²⁺/CaM) kinase inhibitor, such as autocamtide-2-related inhibitory peptide (AIP). In another preferred embodiment, the agent is a diacylglycerol kinase inhibitor such as 6-[2-[4-[(4-fluorophenyl)phenylmethylene]-1-piperidinyl]ethyl]-7-methyl-5H-thiazolo[3,2-a]pyrimidin-5-one (ALX).

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of lithium.

The human cells that may be used in the present method include, but are not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a lithium compound and at least one adjunctive agent.

The lithium compound may be present in an effective amount sufficient to provide a plasma lithium level of 1 mM or less, more preferably, the plasma lithium level is 0.5 mM.

The at least one adjunctive agent used in the method may include, but is not limited to, a mood stabilizer, an anticonvulsant, an antipsychotic, an anxiolytic, and a cholinergic agonist, as described herein.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range. Preferably, the effective amount of lithium is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of lithium.

The mean membrane potential test may further include incubating the cells in vitro in buffer comprising a potential-sensitive dye, resuspending the cells in potential-sensitive dye free-buffer, and measuring the cell fluorescence.

### Eighth Embodiment

The present invention provides a method of optimizing a combination drug treatment therapy for a patient with bipolar disorder (BD), comprising the steps of:

obtaining a ratio of a mean membrane potential that is a mean membrane potential of a first population of cells from the BD patient incubated in vitro in the presence of an agent that alters human calcium-activated potassium channels (hSK₄) activity and in the absence of K⁺, to a mean membrane potential of a second population of the human patient cells incubated in vitro in the absence of the test agent that alters human calcium-activated potassium channels (hSK₄) activity and the presence of K⁺ or absence of K⁺;

comparing the test ratio to (a) and/or (b):
(a) a control ratio of a mean membrane potential of control human cells known to not have said BD incubated in vitro in the presence of the agent that alters human calcium-activated potassium channels hSK₄ and in the absence of K⁺, to a mean membrane potential of the control human cells incubated in vitro in the absence of the agent that alters human calcium-activated potassium channels hSK₄ and in the presence of K⁺ or absence of K⁺,
(b) a bipolar control ratio of a mean membrane potential of bipolar control human cells known to have said BD incubated in vitro in the presence of the agent that alters human calcium-activated potassium channels hSK₄ and in the absence of K⁺, to a mean membrane potential of the bipolar control human cells incubated in vitro in the absence of the agent that alters human calcium-activated potassium channels hSK₄ and in the presence of K⁺ or absence of K⁺;

determining an optimal drug therapy treatment for the BD patient when the ratio of the mean membrane potential obtained is not significantly different from the control ratio in (a), is increased towards the control ratio in comparison to the bipolar control ratio of (b), and/or is significantly higher than the BD control ratio in (b).

The method may further include obtaining an initial ratio of a mean membrane potential from an initial population of cells from the human patient before the obtaining step.

The method may further include optionally modifying at least one drug in the drug therapy treatment for BD when the least one drug treatment therapy for BD is determined to not be the optimal drug therapy treatment. Such as when the ratio of the mean membrane potential obtained is significantly lower than the control ratio of (a), is decreased towards the bipolar control ratio of (b) in comparison to the control ratio of (a), and/or is not significantly different from the BD control ratio of (b).

The agent that may be used include, but is not limited to, a calcium-calmodulin (Ca²⁺/CaM) kinase inhibitor, a diacylglycerol kinase inhibitor, and a PKC inhibitor. Preferably, the agent is a calcium-calmodulin (Ca²⁺/CaM) kinase inhibitor, such as autocamtide-2-related inhibitory peptide (AIP). In another preferred embodiment, the agent is a diacylglycerol kinase inhibitor such as 6-[2-[4-[(4-fluorophenyl)phenylmethylene]-1-piperidinyl]ethyl]-7-methyl-5H-thiazolo[3,2-alpyrimidin-5-one (ALX).

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of lithium.

The human cells that may be used in the present method include, but are not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a lithium compound and at least one adjunctive agent.

The lithium compound may be present in an effective amount sufficient to provide a plasma lithium level of 1 mM or less, more preferably, the plasma lithium level is 0.5 mM.

The at least one adjunctive agent used in the method may include, but is not limited to, a mood stabilizer, an anticonvulsant, an antipsychotic, an anxiolytic, and a cholinergic agonist, as described herein.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range. Preferably, the effective amount of lithium is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of lithium.

The mean membrane potential test may further include incubating the cells in vitro in buffer comprising a potential-sensitive dye, resuspending the cells in potential-sensitive dye free-buffer, and measuring the cell fluorescence.

### Ninth Embodiment

The invention further provides a method of determining an optimum dosage of at least one drug in a combination drug treatment therapy for a patient with BD, that comprises: obtaining a ratio of a mean membrane potential that is a mean membrane potential of a first population of cells from the BD patient incubated in vitro in the presence of an agent that alters human calcium-activated potassium channels (hSK₄) activity and in the absence of K⁺, to a mean membrane potential of a second population of the human patient cells incubated in vitro in the absence of the test agent that alters human calcium-activated potassium channels (hSK₄) activity and the presence of K⁺ or absence of K⁺;

comparing the test ratio to (a) and/or (b):
(a) a control ratio of a mean membrane potential of control human cells known to not have said BD incubated in vitro in the presence of the agent that alters human calcium-activated potassium channels hSK₄ and in the absence of K⁺, to a mean membrane potential of the control human cells incubated in vitro in the absence of the agent that alters human calcium-activated potassium channels hSK₄ and in the presence of K⁺ or absence of K⁺,
(b) a bipolar control ratio of a mean membrane potential of bipolar control human cells known to have said BD incubated in vitro in the presence of the agent that alters human calcium-activated potassium channels hSK₄ and in the absence of K⁺, to a mean membrane potential of the bipolar control human cells incubated in vitro in the absence of the agent that alters human calcium-activated potassium channels hSK₄ and in the presence of K⁺ or absence of K⁺;

determining the dosage of the at least one drug in the combination drug treatment therapy is an optimal dosage for treating BD in the combination therapy when the ratio of the mean membrane potential is not significantly different from the control ratio of (a), is increased towards the control ratio (a) in comparison to or relative to the bipolar control ratio of (b), and/or is significantly higher in comparison to or relative to the bipolar ratio of (b).

The method may further include obtaining an initial ratio of a mean membrane potential from an initial population of cells from the human patient before the obtaining step.

The method optionally further include modifying the dosage of the at least one drug in the drug therapy treatment for BD when the dosage of the at least one drug in the combination therapy is determined to not be the optimal dosage for treating BD based on the mean membrane potential.

The agent that may be used include, but is not limited to, a calcium-calmodulin (Ca²⁺/CaM) kinase inhibitor, a diacylglycerol kinase inhibitor, and a PKC inhibitor. Preferably, the agent is a calcium-calmodulin (Ca²⁺/CaM) kinase inhibitor, such as autocamtide-2-related inhibitory peptide (AIP). In another preferred embodiment, the agent is a diacylglycerol kinase inhibitor such as 6-[2-[4-[(4-fluorophenyl)phenylmethylene]-1-piperidinyl]ethyl]-7-methyl-5H-thiazolo[3,2-alpyrimidin-5-onc (ALX).

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of lithium.

The human cells that may be used in the present method include, but are not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a lithium compound and at least one adjunctive agent.

The lithium compound may be present in an effective amount sufficient to provide a plasma lithium level of 1 mM or less, more preferably, the plasma lithium level is 0.5 mM.

The at least one adjunctive agent used in the method may include, but is not limited to, a mood stabilizer, an anticonvulsant, an antipsychotic, an anxiolytic, and a cholinergic agonist, as described herein.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range. Preferably, the effective amount of lithium is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of lithium.

The mean membrane potential test may further include incubating the cells in vitro in buffer comprising a potential-sensitive dye, resuspending the cells in potential-sensitive dye free-buffer, and measuring the cell fluorescence.

### Tenth Embodiment

The present invention further provides a method for monitoring the efficacy of a combination drug treatment therapy for the treatment of bipolar disorder (BD), said method comprising:

obtaining a ratio of a mean membrane potential that is a mean membrane potential of a first population of cells from the BD patient incubated in vitro in the presence of an agent that alters human calcium-activated potassium channels (hSK₄) activity and in the absence of K⁺, to a mean membrane potential of a second population of the human patient cells incubated in vitro in the absence of the test agent that alters human calcium-activated potassium channels (hSK₄) activity and the presence of K⁺ or absence of K⁺;

comparing the test ratio to (a) and/or (b):
(a) a control ratio of a mean membrane potential of control human cells known to not have said BD incubated in vitro in the presence of the agent that alters human calcium-activated potassium channels hSK₄ and in the absence of K⁺, to a mean membrane potential of the control human cells incubated in vitro in the absence of the agent that alters human calcium-activated potassium channels hSK₄ and in the presence of K⁺ or absence of K⁺,
(b) a bipolar control ratio of a mean membrane potential of bipolar control human cells known to have said BD incubated in vitro in the presence of the agent that alters human calcium-activated potassium channels hSK₄ and in the absence of K⁺, to a mean membrane potential of the bipolar control human cells incubated in vitro in the absence of the agent that alters human calcium-activated potassium channels hSK₄ and in the presence of K⁺ or absence of K⁺;

determining the combination drug treatment therapy is efficacious based on the mean membrane potential when the ratio of the mean membrane potential obtained is not significantly different from the control ratio of (a), is increased towards the control ratio (a) in comparison to or relative to the bipolar control ratio of (b), and/or is significantly higher in comparison to or relative to the bipolar ratio of (b).

The method may further include obtaining an initial ratio of a mean membrane potential from an initial population of cells from the human patient before the obtaining step.

The method optionally further include determining the combination drug treatment therapy is not efficacious based on the mean membrane potential when the ratio of the mean membrane potential obtained is determined to not be efficacious based on the mean membrane potential. Such as when the ratio of the mean membrane potential obtained is lower in comparison to or relative to the control ratio of (a), is decreased towards the bipolar control ratio of (b) in comparison to or relative to the control ratio (a), and/or is not significantly different from the BD control ratio of (b).

The method may optionally further include adjusting a dosage of one or more agents in the combination drug treatment therapy when the combination therapy is determined to not be efficacious based on the mean membrane potential.

The agent that may be used include, but is not limited to, a calcium-calmodulin (Ca²⁺/CaM) kinase inhibitor, a diacylglycerol kinase inhibitor, and a PKC inhibitor. Preferably, the agent is a calcium-calmodulin (Ca²⁺/CaM) kinase inhibitor, such as autocamtide-2-related inhibitory peptide (AIP). In another preferred embodiment, the agent is a diacylglycerol kinase inhibitor such as 6-[2-[4-[(4-fluorophenyl)phenylmethylene]-1-piperidinyl]ethyl]-7-methyl-5H-thiazolo[3,2-alpyrimidin-5-one (ALX).

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range.

Preferably, the effective amount of lithium is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy. Preferably, the effective amount of an adjunctive agent is the dosage amount that is sufficient to improve or enhance the therapeutic effect or therapeutically efficacious plasma level of lithium.

The human cells that may be used in the present method include, but are not limited to, red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells. Preferably, the human cells are selected from the group consisting of red blood cells and lymphoblasts.

The combination drug treatment therapy of the present invention is a synergistic combination.

The combination drug treatment therapy may comprise a lithium compound and at least one adjunctive agent.

The lithium compound may be present in an effective amount sufficient to provide a plasma lithium level of 1 mM or less, more preferably, the plasma lithium level is 0.5 mM.

The at least one adjunctive agent used in the method may include, but is not limited to, a mood stabilizer, an anticonvulsant, an antipsychotic, an anxiolytic, and a cholinergic agonist, as described herein.

Preferably, the effective amount of lithium compound is a dose amount that is less than a dosage of lithium required to provide a therapeutic effect for BD therapy when used alone, or is a dose amount that is less than a dosage of lithium required to provide a therapeutically efficacious plasma lithium level for BD therapy when used alone. For instance, the effective dose may be a dose that brings the diagnostic probability to the negative range. Preferably, the effective amount of lithium is the dosage amount that improves or enhances the therapeutic effect or therapeutically efficacious plasma level of an adjunctive agent.

Preferably, the effective amount of an adjunctive agent in the drug combination of the present invention, is an amount less than that used in its current drug therapy.

Preferably, the effective amount of an adjunctive agent is the dosage amount that is sufficient to improve or enhance the therapeutic effect or therapeutically efficacious plasma level of lithium.

The mean membrane potential test may further include incubating the cells in vitro in buffer comprising a potential-sensitive dye, resuspending the cells in potential-sensitive dye free-buffer, and measuring the cell fluorescence.

### Eleventh Embodiment

In some embodiments thereof, the method includes the steps of:
treating the BD patient with a dosage of a combination therapy for treating BD;
obtaining at least one sample from the patient which is collected after the treating step;
performing on each sample, a mean membrane potential test including obtaining a ratio of a mean membrane potential from a first population of cells from the sample incubated in vitro in the presence of a compound that alters Na⁺K⁺ ATPase activity and in the absence of K⁺, to a mean membrane potential from a second population of cells from the sample incubated in vitro in the absence of the compound that alters Na⁺K⁺ ATPase activity and in the presence or absence of K+,
comparing the ratio of the mean membrane potential to (a) and/or (b) wherein (a) is a control ratio of a mean membrane potential of control human cells known to not have BD incubated in vitro in the presence of the compound that alters Na⁺K⁺ ATPase activity and in the absence of K⁺, to a mean membrane potential of the control human cells incubated in vitro in the absence of the compound that alters Na⁺K⁺ ATPase activity and in the presence or absence of K⁺, and (b) is a BD control ratio of a mean membrane potential of BD control human cells known to have BD incubated in vitro in the presence of the compound that alters Na⁺K⁺ ATPase activity and in the absence of K⁺, to a mean membrane potential of the BD control human cells incubated in vitro in the presence of the compound that alters Na⁺K⁺ ATPase activity and in the presence or absence of K⁺;
modifying the drug dosage based on the mean membrane potential test; and
identifying an optimal drug dosage for treating the human patient when the ratio of the mean membrane potential obtained is not significantly different from the control ratio of (a), is increased towards the control ratio (a) in comparison to or relative to the bipolar control ratio (h), and/or is significantly higher in comparison to or relative to the BD control ratio in (b).

The ratio of the mean membrane potential obtained may be not significantly different from or relative to the control ratio of (a), significantly increased towards the control ratio (a) in comparison to or relative to the bipolar control ratio (b), and/or is significantly higher in comparison to or relative to the BD control ratio in (b).

When used with the combination therapies of the present invention, these methods for determining the optimum dose can be used to even further reduce the possibility of side effects.

In other aspects, the present invention relates to monitoring the efficacy of a combination therapy for the treatment of BD, by analyzing the membrane potential of cells isolated from a BD patient treated with the combination therapy, and calculating a membrane potential ratio therefrom. In some embodiments thereof, the method includes the steps of:
treating the BD patient with a dosage of a combination therapy for treating BD;
obtaining at least one sample from the patient which is collected after the treating step;
performing on each sample, a mean membrane potential test including obtaining a ratio of a mean membrane potential from a first population of cells from the sample incubated in vitro in the presence of a compound that alters Na⁺K⁺ ATPase activity and in the absence of K⁺, to a mean membrane potential from a second population of cells from the sample incubated in vitro in the absence of the compound that alters Na⁺K⁺ ATPase activity and in the presence or absence of K+,
comparing the ratio of the mean membrane potential to (a) and/or (b) wherein (a) is a control ratio of a mean membrane potential of control human cells known to not have BD incubated in vitro in the presence of the compound that alters Na⁺K⁺ ATPase activity and in the absence of K⁺, to a mean membrane potential of the control human cells incubated in vitro in the absence of the compound that alters Na⁺K⁺ ATPase activity and in the presence or absence of K⁺, and (b) is a BD control ratio of a mean membrane potential of BD control human cells known to have BD incubated in vitro in the presence of the compound that alters Na⁺K⁺ ATPase activity and in the absence of K⁺, to a mean membrane potential of the BD control human cells incubated in vitro in the presence of the compound that alters Na⁺K⁺ ATPase activity and in the presence or absence of K⁺;
determining whether the drug dosage is efficacious based on the mean membrane potential test; and
optionally, adjusting the dosage of one or more agents in the combination therapy when the ratio of the mean membrane potential obtained is significantly lower in comparison to or relative to the control ratio of (a) and/or is not different from or relative to the BD control ratio of (b).

When used with the combination therapies of the present invention, these monitoring methods can be used to maintain efficacy, while reducing the possibility of side effects.

In some embodiments, the methods of the present invention further include obtaining an initial ratio of a mean membrane potential from an initial population of cells from the BD patient before the treatment step.

The phorbol ester according to the present invention include phorbol 12-myristate 13-acetate (PMA), 12-O-tetradecanoylphorbol 13-acetate, phorbol 12-myristate 13-acetate 4-O-methyl ether, phorbol 12,13-dibutyrate (PDBu), phorbol 12,13-didecanoate (PDD), and phorbol 12,13-dinonanoate 20-homovanillate.

In another embodiment, a compound that decreases the density and/or activity of the potassium channel may be used in the therapy optimization and monitoring methods according to the present invention. For example, low concentrations of ouabain may be useful in determining the effect of the BD treatment using MPR™.

Potassium-containing buffers that may be used in the therapy optimization and monitoring methods according to the present invention can be created by adding potassium to the buffers shown in the table above that do not contain potassium. Potassium-containing buffers useful in the methods according to the present invention preferably have a K⁺ concentration in the range of approximately 2 mM to 7 mM, more preferably have a K⁺ concentration of approximately 5 mM, and still more preferably have a K⁺ concentration of 5 mM.

The K⁺-containing buffer may be, for example, a HEPES buffer to which potassium has also been added (5 mMKCl, 4 mMNaHCO₃, 5 mMHEPES, 134 mMNaCl, 2.3 mMCaCl₂, and 5 mM glucose; pH 7.3-7.5, preferably 7.4), and which may be referred to as “regular” or “stock” or “reference” buffer. The K⁺-free buffer used in the examples is a HEPESbuffer without potassium (4 mMNaHCO₃, 5 mMHEPES, 134 mMNaCl, 2.3 mMCaCl₂, and 5 mM glucose; pH 6.6-7.0, preferably 6.8), and is also referred to as “test” buffer.

The membrane potential of a BD patient's cells, for the therapy optimization and monitoring methods according to the present invention, may also be ascertained, or confirmed, by any conventional method, such as by examining the fluorescence intensity of a potential-sensitive lipophilic fluorescent dye. The membrane potential is directly proportional to the intensity of fluorescence according to the following equation: I=CV, wherein I is the fluorescence intensity of a lipophilic fluorescent dye. V is the voltage or membrane potential, and C is a constant that can vary depending on a number of factors such as, but not limited to, temperature, lamp intensity, number of cells, concentration of the fluorescent dye, incubation time, and lipid composition of cells used. The calibration and determination of the value for C can be a cumbersome and unreliable procedure. Thus, in some embodiments, by using the ratio of the fluorescence intensity (I₁) of one sample of cells to the fluorescence intensity (I₂) of another sample of cells, the constant (C) is canceled out. Such ratio-metric measurements are preferred over absolute measurements.

Examples of potential-sensitive dyes that may be adapted for use in the present invention, along with their charges and optical responses, are shown below in Table 3 (all available from Molecular Probes Inc., Eugene, Oreg., US).

TABLE 1

| | Structure | |
|---|---|---|
| Dye | (Charge) | Optical Response |
| DiOC₂(3) | Carbocyanine | Slow; fluorescence response to depolarization |
| DiOC₅(3) | (cationic) | depends on staining concentration and detection |
| DiOC₆(3) | | method. |
| DiSC₃(5) | | |
| DiIC₁(5) | | |
| JC-1 | Carbocyanine | Slow; fluorescence emission ratio 585/520 nm |
| JC-9 | (cationic) | increases upon membrane hyperpolarization. |
| Tetramethyl-rhodamine | Rhodamine | Stow; used to obtain unbiased images of |
| methyl and ethyl esters | (cationic) | potential-dependent dye distribution. |
| Rhodamine 123 | | |
| Oxonol V | Oxonol (anionic) | Slow; fluorescence decreases upon membrane |
| Oxonol VI | | hyperpolarization. |
| DiBAC₄(3) | Oxonol (anionic) | Slow; fluorescence decreases upon membrane |
| DiBAC₄(5) | | hyperpolarization. |
| DiSBAC₂(3) | | |
| Merocyanine 540 | Merocyanine | Fast/Slow (biphasic response). |

Indo-(DiI), thia-(DiS) and oxa-(DiO) carbocyanines with short alkyl tails (<7 carbon atoms) were among the first potentiometric fluorescent probes developed. These cationic dyes accumulate on hyperpolarized membranes and are translocated into the lipid bilayer. DiOC₆(3) (3,3′-dihexyloxacarbocyanine iodide), a cell-permeant, voltage sensitive, green-fluorescent dye, has been the most widely used carbocyanine dye for membrane potential measurements, followed closely by DiOC₅(3) (3,3′-dipentyloxacarbocyanine iodide). Thus, in a preferred embodiment of the methods according to the present invention, membrane potentials may be measured using DiOC₆(3) in conjunction with a fluorescence spectrometer.

In one embodiment, the cells are incubated in the presence of K⁺. In another embodiment, the cells are incubated in the absence of K⁺. As used herein. “presence of K⁺” preferably means a K⁺ concentration in the range of approximately 2 mM to 7 mM, preferably approximately 5 mM.

The therapy optimization and monitoring methods according to the present invention may be used with any cell type, such as, but not limited to, erythrocytes, platelets, leukocytes, macrophages, monocytes, dendritic cells, fibroblasts, epidermal cells, mucosal tissue cells, cells in the cerebrospinal fluid, and hair cells. Cells present in blood, skin cells, hair cells, or mucosal tissue cells may be more convenient to use because of the ease of harvesting these cell types.

### Twelfth Embodiment

In a twelfth embodiment, the present invention provides a pharmaceutical combination comprising a lithium compound and at least one adjunctive agent, as well as a pharmaceutical composition comprising a lithium compound and at least one adjunctive agent; and a pharmaceutically acceptable carrier.

The effective amount of lithium compound of the pharmaceutical combination or composition may be a dose amount that is less than a dosage of lithium required to provide a therapeutically efficacious plasma lithium level for BD therapy when used alone. Preferably, the dose provides a plasma lithium level of 1 mM or less. More preferably, the dose provides a plasma lithium level of 0.5 mM or less.

The at least one adjunctive agent of the pharmaceutical combination or composition may be administered at a dose that is less than a dosage of the at least one adjunctive agent required to provide a therapeutically efficacious plasma level of the at least one adjunctive agent when administered alone.

The at least one adjunctive agent of the pharmaceutical combination or composition may include, but is not limited to, a mood stabilizer, an anticonvulsant, an antipsychotic, an anxiolytic, and a cholinergic agonist, as described herein.

Preferably, the at least one adjunctive agent of the pharmaceutical combination or composition is a cholinergic agonist including, hut not limited to, donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof. Preferably, the cholinergic agonist is carbachol and pharmaceutically acceptable salts thereof, or donepezil and pharmaceutically acceptable salts thereof.

Carbachol may be administered to provide a plasma concentration of 10 μM or less. Donepezil may be administered to said patient to provide a plasma concentration of 10 ng/ml or less.

Preferably, the at least one adjunctive agent of the pharmaceutical combination or composition is an antipsychotic including, but not limited to, haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof. Preferably, the antipsychotic is clozapine and pharmaceutically acceptable salts thereof.

Clozapine may be administered to provide a plasma concentration of 100 ng/ml or less.

### Thirteenth Embodiment

The present invention also provides the following kits.

A kit that may include (a) a K⁺-containing HEPES reference buffer; (b) a K⁺-free HEPES buffer; (c) a potential-sensitive dye; and (d) instructions for performing an assay to determine an optimal combination drug treatment therapy for bipolar disorder.

A kit that may include (a) a K⁺-containing HEPES reference buffer; (b) a K⁺-free HEPES buffer; (c) a potential-sensitive dye; and (d) instructions for performing an assay to optimize a combination drug treatment therapy for bipolar disorder.

A kit that may include (a) a K⁺-containing HEPES reference buffer; (b) a K⁺-free HEPES buffer; (c) a potential-sensitive dye; and (d) instructions for performing an assay to determine an optimum dosage of a drug in combination drug treatment therapy for bipolar disorder.

A kit that may include (a) a K⁺-containing HEPES reference buffer; (b) a K⁺-free HEPES buffer; (c) a potential-sensitive dye; and (d) instructions for performing an assay to monitor the efficacy of a combination drug treatment therapy for bipolar disorder.

### EXAMPLES

The following examples are provided for illustrative purposes only and are in no way intended to limit the scope of the invention.

### Example 1: Administering Carbachol with Lithium Reduces the Dose of Lithium Needed to be Therapeutic

Carbachol, a choline carbamate, is a cholinergic agonist. At present, carbachol is primarily used in the form of an ophthalmic solution for treating various ophthalmic conditions, such as glaucoma; or for use during ophthalmic surgery. Using the MPR™ test assay described previously by Thiruvengadam (U.S. Pat. No. 7,425,410, incorporated by reference herein in its entirety), the effect of carbachol in combination with lithium on the MPR™ was determined. As mentioned herein, MPR™ is the ratio between the membrane potential (MP) in the test buffer and that in the reference buffer. In these experiments, the reference buffer contained NaCl, CaCl₂, glucose and HEPES, whereas the test buffer contained ethyl alcohol (EtOH) in addition to NaCl, CaCl₂, glucose and HEPES. Lithium, inositol and carbachol were added to the test buffer in these experiments.

Whole blood samples were obtained from BD patients, and a portion from each blood sample was suspended in the test buffer for 20 minutes, and a portion from each blood sample was suspended in the reference buffer for 20 minutes. After this incubation, the samples were centrifuged for five minutes, drained, then re-suspended in their respective buffer (test or reference buffer). These samples were then distributed in 96 well plates, and tested in a plate reader (FLx 800 manufactured by BioTek).

As shown in FIG. 1, the MPR™ value for 1 mM Li was 0.814. However, when 0.5 mM Li, 2.5 μM inositol and 10 μM carbachol were used, the MPR™ value improved to 0.860. (Carbachol is not a psychiatric drug although it is used for the eye. https://www.drugs.com/dosage/carbachol-ophthalmic.html) (Applies to the following strength(s): 0.01%0.75% 1.5%2.25%3%). (Instill no more than 0.5 mL into the anterior chamber of the affected eye(s) for the production of miosis during ocular surgery.) Thus, this experiment showed that the MPR™ value obtained with lithium alone, at a concentration of 1 mM, can be significantly improved even at half the dose of lithium (0.5 mM Li), when it is used in combination with what would otherwise be a sub-therapeutic dose of carbachol. This demonstrates the synergistic effect obtained with the combination of lithium and carbachol.

### Example 2: Administering Clozapine with Lithium Reduces the Dose of Lithium Needed to be Therapeutic

Clozapine was discovered in the 1960s, and is a dibenzodiazepine used in mental healthcare. It was the first atypical antipsychotic. Clozapine is also a cholinergic agonist. Clozapine has been used to treat BD (Calabrese et al. “Clozapine for Bipolar Disorder, Letter to the Editor.” Am. J. Psychiatry, 2000, 157: 9; Calabrese et al. “Clozapine for treatment-refractory mania.” Am. J. Psychiatry, 1996, 153: 759-764; Frye et al. “Clozapine in Bipolar Disorder: Treatment Implications for Atypical Antipsychotics.” J. Affec. Disord., 1998, 48: 91-104; and Vangala et al. “Clozapine Associated with Decreased Suicidality in Bipolar Disorder: A Case Report,” Bipolar Disord., 1999, 2: 123-124).

However, the side effects of clozapine are significant at presently used therapeutic levels (ranging from 200-1000 ng/ml of blood plasma, see Freudenreich et al. “Clozapine Drug Levels Guide Dosing,” Current Psychiatry, 2009, 8(3)). Using the MPR™ test assay, the effect of clozapine in combination with lithium on the MPR™ was determined. In these experiments, the reference buffer contained NaCl, CaCl₂, glucose and HEPES, whereas the test buffer contained ethyl alcohol (EtOH) in addition to NaCl, CaCl₂, glucose and HEPES. Lithium, inositol and clozapine were added to the test buffer in these experiments.

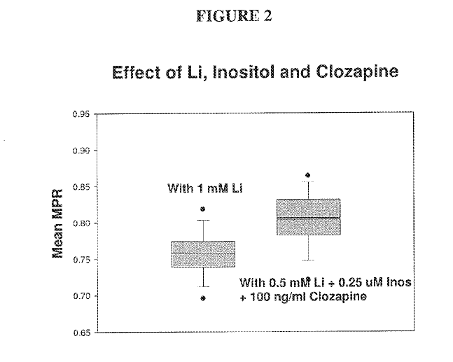

Whole blood samples were obtained from BD patients, and a portion from each blood sample was suspended in the test buffer for 20 minutes, and a portion from each blood sample was suspended in the reference buffer for 20 minutes. After this incubation, the samples were centrifuged for five minutes, drained, then re-suspended in their respective buffer (test or reference buffer). These samples were then distributed in 96 well plates, and tested in a plate reader (FLx 800 manufactured by BioTek). The results are depicted in FIG. 2.

As shown in FIG. 2, the MPR™ value for 1 mM Li was 0.757. However, when 0.5 mM Li, 2.5 μM inositol and 100 ng/ml clozapine were used, the MPR™ value improved to 0.804. Thus, this experiment showed that the MPR™ value obtained with lithium alone, at a concentration of 1 mM, can be significantly improved even at half the dose of lithium (0.5 mM Li), when it is used in combination with what would otherwise be a sub-therapeutic dose of clozapine. This demonstrates the synergistic effect obtained with the combination of lithium and clozapine.

### Example 3: Administering Donepezil with Lithium Reduces the Dose of Lithium Needed to be Therapeutic

Donepezil is used to improve the cognition and behavior of patients with Alzheimer's disease. Donepezil is a centrally-acting reversible acetylcholinesterase inhibitor. The therapeutic reference range for donepezil is 30-75 ng/ml, see Hefner et al. (“Monitoring (TDM) of donepezil in patients with Alzheimer's dementia,” Pharmacopsychiatry, 2013, 46: A42).

Using the MPR™ test assay, the effect of donepezil in combination with lithium on the MPR™ was determined. In these experiments, the reference buffer contained NaCl, CaCl₂, glucose and HEPES, whereas the test buffer contained ethyl alcohol (EtOH) in addition to NaCl, CaCl₂, glucose and HEPES. Lithium, inositol and donepezil were added to the test buffer in these experiments.

Whole blood samples were obtained from BD patients, and a portion from each blood sample was suspended in the test buffer for 20 minutes, and a portion from each blood sample was suspended in the reference buffer for 20 minutes. After this incubation, the samples were centrifuged for five minutes, drained, then re-suspended in their respective buffer (test or reference buffer). These samples were then distributed in 96 well plates, and tested in a plate reader (FLx 800 manufactured by BioTek). The results are depicted in FIG. 2.

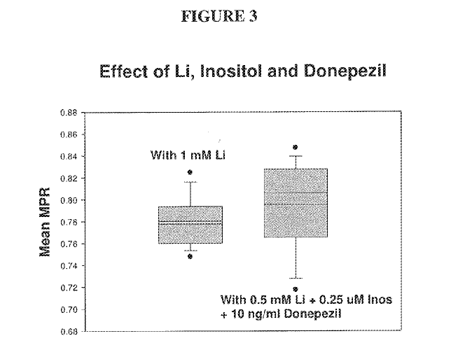

As shown in FIG. 3, the MPR™ value for 1 mM Li was 0.780. However, when 0.5 mM Li, 2.5 μM inositol and 10 ng/ml donepezil were used, the MPR™ value improved to 0.796. Thus, this experiment showed that the MPR™ value obtained with lithium alone, at a concentration of 1 mM, can be significantly improved even at half the dose of lithium (0.5 mM Li), when it is used in combination with what would otherwise be a sub-therapeutic dose of donepezil (10 ng/ml, as compared to the therapeutic reference range of 30-75 ng/ml). This demonstrates the synergistic effect obtained with the combination of lithium and donepezil.

### Example 4: In BD Patient MPR™ Returns to Negative with Treatment

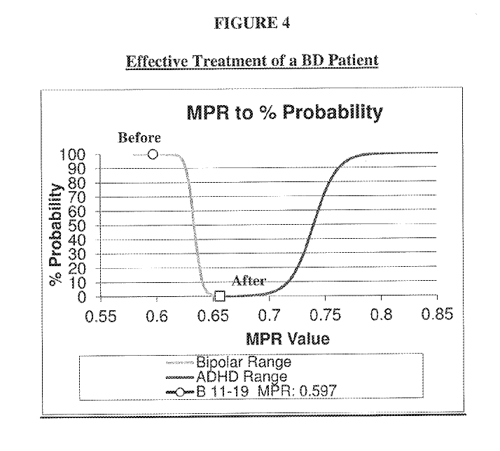

A 43 year old woman, who had a long standing diagnosis of BD and comorbid difficulties with alcohol from well before the start of her treatment. At the time of her first blood draw, she reported having been off of her medications for BD for one month, but had not become clinically symptomatic, and tested in the BD range (“(Before)” of FIG. 4). When retested, she had been taking lithium, quetiapine, and mirtazapine for some time and had been back at work. The result obtained was in the negative range (“(After)” of FIG. 4) when she was reasonably stable and taking medication appropriate for her diagnosis. Subsequently, she reported anxiety, occurring while she continued taking lithium, quetiapine, and mirtazapine. She thought she might be experiencing a return of bipolar symptomatology, but this was determined to possibly be situational instead. She had some awareness of issues with her fiancé and this awareness was “bolstered” by the notion that her MPR™ test had been in the normal range on her medication regimen not so many months before. Time limited couples therapy was suggested. She wanted a medication to help her through what she was experiencing, but could not be given benzodiazepines because of a history of difficulty with alcohol. Very low dosage perphenazine was given, in keeping with its old indication for neurotic anxiety. This was very quickly discontinued by her because of blurred vision. The result in the negative range helped in the decision to essentially stay the course with the medications associated with that result.

The Examples demonstrate the synergistic effect of using carbachol, clozepine and/or donepezil, in combination with lithium; and show that superior treatment effects can be obtained, as compared to the cholinergic agonist or lithium alone, with what would otherwise be sub-therapeutic doses of these agents.

All references, including publications, patent applications, and patents cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed clement as essential to the practice of the invention.

What is claimed is:

1. A method of determining an optimal combination drug treatment therapy for a patient with bipolar disorder (BD), comprising:
obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K⁺ or absence of K⁺;
comparing the ratio of the mean membrane potential to (a) and/or (b):
(a) a control ratio of a mean membrane potential of first population of control human cells known to not have BD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+,
(b) a bipolar control ratio of a mean membrane potential of first population of bipolar control human cells known to have BD incubated in vitro in the presence of the agent that alters diacylglycerol signaling and in the absence of K+, to a mean membrane potential of a second population of the bipolar control human cells incubated in vitro in the absence of the agent that alters diacylglycerol signaling and in the presence of K+ or absence of K+:
identifying the optimal combination drug treatment therapy when the ratio of the mean membrane potential obtained is not significantly different from the control ratio in (a), is increased towards the control ratio in comparison to the bipolar control ratio of (b), and/or is significantly higher than the BD control ratio in (b).

2. A method of optimizing a combination drug treatment therapy for a patient with bipolar disorder (BD), comprising the steps of:
obtaining at least one sample from a BD patient in a drug therapy treatment for BD;
performing on each sample, a mean membrane potential test comprising:
obtaining a ratio of a mean membrane potential that is a mean membrane potential of a first population of cells from the sample incubated in vitro in the presence of an agent that alters diacylglycerol signaling and in the absence of K⁺ to a mean membrane potential of a second population of the sample incubated in vitro in the absence of the test agent that alters diacylglycerol signaling and in the presence of K⁺ or absence of K⁺;
comparing the ratio of the mean membrane potential to (a) and/or (b):
(a) a control ratio of a mean membrane potential of a first population of control human cells known to not have BD incubated in vitro in the presence of the agent that alters diacylglycerol signaling and in the absence of K+, to a mean membrane potential of a second population of the control human cells incubated in vitro in the absence of the agent that alters diacylglycerol signaling and in the presence of K+ or absence of K+,
(b) a bipolar control ratio of a mean membrane potential of a first population of bipolar control human cells known to have BD incubated in vitro in the presence of the agent that alters diacylglycerol signaling and in the absence of K+, to a mean membrane potential of a second population of the bipolar control human cells incubated in vitro in the absence of the agent that alters diacylglycerol signaling and in the presence of K+ or absence of K+:
determining an optimal drug therapy treatment for the BD patient based on the mean membrane potential test when the ratio of the mean membrane potential obtained is not significantly different from the control ratio of (a), is increased towards the control ratio in comparison to the bipolar control ratio of (b), and/or is significantly higher than the BD control ratio of (b); and
optionally, modifying at least one drug in the drug therapy treatment for BD when the least one drug treatment therapy for BD is determined to not be the optimal drug therapy treatment based on the mean membrane potential test.

3. A method for determining an optimum dosage of a drug in a combination drug treatment therapy for the treatment of bipolar disorder (BD), said method comprising:
obtaining at least one sample from a BD patient treated with a dosage of a drug in a combination therapy;
performing on each sample, a mean membrane potential test comprising:
obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K⁺ or absence of K⁺;
comparing the ratio of the mean membrane potential to (a) and/or (b):
(a) a control ratio of a mean membrane potential of a first population of cells from a control human known to not have said BD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+,
(b) a bipolar control ratio of a mean membrane potential of a first population of cells from a bipolar control human known to have said BD incubated in vitro in the presence of the agent that alters diacylglycerol signaling and in the absence of K+, to a mean membrane potential of a second population of cells from the bipolar control human incubated in vitro in the absence of the agent that alters diacylglycerol signaling and in the presence of K+ or absence of K+;
determining the dosage of the drug in the combination drug treatment therapy is an optimal dosage for treating BD in the combination therapy based on the mean membrane potential test when the ratio of the mean membrane potential obtained is not significantly different from the control ratio of (a), is increased towards the control ratio in comparison to the bipolar control ratio of (b), and/or is significantly higher than the BD control ratio of (b); and
optionally, modifying the dosage of the drug in the combination drug treatment therapy when the dosage of the drug in the combination therapy is determined to be not the optimal dosage for treating BD based on the mean membrane potential test.

4. A method for monitoring the efficacy of a combination drug treatment therapy for the treatment of bipolar disorder (BD), said method comprising:
obtaining at least one sample from a BD patient treated with a combination drug treatment therapy for treating BD;
performing on each sample, a mean membrane potential test comprising:
obtaining a ratio of a mean membrane potential that is a mean membrane potential of a first population of cells from the BD patient incubated in vitro in the presence of an agent that alters diacylglycerol signaling and in the absence of K⁺, to a mean membrane potential of a second population of cells from the BD patient incubated in vitro in the absence of the test agent that alters diacylglycerol signaling and in the presence of K⁺ or absence of K⁺;
comparing the ratio of the mean membrane potential to (a) and/or (b):
(a) a control ratio of a mean membrane potential of a first population of cells from a control human known to not have said BD incubated in vitro in the presence of the agent that alters diacylglycerol signaling and in the absence of K+, to a mean membrane potential of a second population of cells from the control human incubated in vitro in the absence of the agent that alters diacylglycerol signaling and in the presence of K+ or absence of K+,
(b) a bipolar control ratio of a mean membrane potential of a first population of cells from a bipolar control human known to have said BD incubated in vitro in the presence of the agent that alters diacylglycerol signaling and in the absence of K+, to a mean membrane potential of a second population of cells from the bipolar control human incubated in vitro in the absence of the agent that alters diacylglycerol signaling and in the presence of K+ or absence of K+;
determining the combination drug treatment therapy is efficacious based on the mean membrane potential test when the ratio of the mean membrane potential obtained is not significantly different from the control ratio in (a), is increased towards the control ratio in comparison to the bipolar control ratio of (b), and/or is significantly higher than the BD control ratio in (b); and
optionally, adjusting a dosage of one or more agents in the combination drug treatment therapy when the combination therapy is determined to be not efficacious based on the mean membrane potential test.

5. The method according to claim 1, 2, 3, or 4, further comprising obtaining an initial ratio of a mean membrane potential from an initial population of cells from the human patient before the obtaining step.

6. The method of claim 1, 2, 3, or 4, wherein the human cells is selected from the group consisting of red blood cells, lymphoblasts, erythocytes, platelets, leukocytes, macrophages, monocytes, dendritic cells, fibroblasts, epidermal cells, mucosal tissue cells, cells of cerebrospinal fluid, hair cells, and whole blood cells.

7. The method of claim 6, wherein the human cells is selected from the group consisting of red blood cells and lymphoblasts.

8. The method of claim 1, 2, 3, or 4, wherein the combination drug treatment therapy is synergistic combination.

9. The method of claim 8, wherein the combination drug treatment therapy comprises a lithium compound and at least one adjunctive agent.

10. The method of claim 9, wherein the lithium compound is present in an effective amount sufficient to provide a plasma lithium level of 1 mM or less.

11. The method of claim 10, wherein the plasma lithium level is 0.5 mM or less.

12. The method of claim 9, wherein the at least one adjunctive agent is selected from the group consisting of a mood stabilizer, an anticonvulsant, an antipsychotic, an anxiolytic, and a cholinergic agonist.

13. The method of claim 12, wherein the cholinergic agonist is selected from the group consisting of donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof.

14. The method of claim 12, wherein the mood stabilizer is selected from the group consisting of valproate, divalproex, carbamazepine, lamotrigine, oxcarbazepine, and pharmaceutically acceptable salts thereof.

15. The method of claim 12, wherein the anticonvulsant is selected from the group consisting of lamotrigine, perampanel, mephobarbital, primidone, phenobarbital, diazepam, clonazepam, lorazepam, clobazam, felbamate, topiramate, acetazolamide, zonisamide, rufinamide, oxcarbazepine, carbamazepine, eslicarbazepine, valproic acid, divalproex sodium, gabapentin, gabapentin enacarbil, tiagabine, phenytoin, fosphenytoin, mephenytoin, ethotoin, magnesium sulfate, lacosamide, ezogabine, trimethadione, levetiracetam, ethosuximide, methsuximide, and pharmaceutically acceptable salts thereof.

16. The method of claim 12, wherein the antipsychotic is selected from the group consisting of haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof.

17. The method of claim 12, wherein the antidepressant is selected from the group consisting of fluoxetine, ariprazole, doxepin, clomipramine, bupropion, amoxapine, nortriptyline, vortioxetine, citalopram, duloxetine, trazodone, venlafaxine, selegiline, perphenazine, amitriptyline, levomilnacipram, desvenlafaxine, lurasidone, lamotrigine, escitalopram, chlordiazepoxide, isocarboxazid, phenelzine, desipramine, trazodone, tranylcypromine, paroxetine, mirtazapine, quetiapine, nefazodone, doxepin, trimipramine, imipramine, vilazodone, protriptyline, sertraline, olanzapine, and pharmaceutically acceptable salts thereof.

18. The method of claim 12, wherein the anxiolytic is selected from the group consisting of secobarbital, mephobarbital, pentobarbital, phenobarbital, amobarbital, butabarbital, estazolam, alprazolam, flurazepam, diazepam, chlordiazepoxide, clorazepate, clonazepam, oxazepam, diazepam, triazolam, lorazepam, temazepam, midazolam, clobazam, diphenhydramine, zolpidem, chloral hydrate, doxepin, sodium oxybate, doxylamine, doxepin, hydroxyzine, meprobamate, ethchlorvynol, eszopiclone, buspirone, zalephon, ramelteon, suvorexant, tryptophan, tasimelteon, dexmedetomidine, and pharmaceutically acceptable salts thereof.

19. The method of claim 1, 2, 3, or 4, wherein the agent that alters diacylglycerol signaling is selected from the group consisting of a calcium-calmodulin (Ca²⁺/CaM) kinase inhibitor, a diacylglycerol kinase inhibitor, a protein kinase C inhibitor, and an agent that affects calcium-activated potassium (CaK) channels.

20. The method of claim 19, wherein the agent is a calcium-calmodulin (Ca²⁺/CaM) kinase inhibitor.

21. The method of claim 20, wherein the calcium-calmodulin (Ca²⁺/CaM) kinase inhibitor is autocamtide-2-related inhibitory peptide (AIP).

22. The method of claim 19, wherein the agent is a diacylglycerol kinase inhibitor.

23. The method of claim 22, wherein the diacylglycerol kinase inhibitor is 6-[2-[4-[(4-fluorophenyl)phenylmethylene]-1-piperidinyl]ethyl]-7-methyl-5H-thiazolo[3,2-alpyrimidin-5-one (ALX).

24. The method of claim 1, 2, 3, or 4, wherein the mean membrane potential test further comprises incubating the cells in vitro in buffer comprising a potential-sensitive dye, resuspending the cells in potential-sensitive dye free-buffer, and measuring the cell fluorescence.

25. The method of claim 1, 2, 3, or 4, wherein the agent that alters K⁺ channel activity is ethanol, amphetamine, ephedrine, cocaine, caffeine, nicotine, methylphenidate, lithium, δ-9-tetrahydrocannibinol, phencyclidine, lysergic acid diethylamide (LSD), mescaline, or combinations thereof.

26. The method of claim 25, wherein the agent that alters K⁺ channel activity is ethanol.

27. A method of treating bipolar disorder (BD), comprising administering an effective amount of a lithium compound and at least one adjunctive agent to a human patient with BD.

28. A method of increasing the therapeutic efficacy of a lithium compound for the treatment of bipolar disorder (BD), comprising administering an effective amount of a lithium compound with at least one adjunctive agent, to a human patient with BD.

29. The method of claim 27 or 28, wherein the at least one adjunctive agent and the lithium compound form a synergistic combination or composition to treat said BD.

30. The method of claim 27 or 28, wherein the effective amount of lithium compound is a dose amount that is less than a dosage of lithium required to provide a therapeutically efficacious plasma lithium level for BD therapy when used alone.

31. The method of claim 30, wherein the dose provides a plasma lithium level of 1 mM or less.

32. The method of claim 31, wherein the dose provides a plasma lithium level of 0.5 mM or less.

33. The method of claim 27 or 28, wherein the at least one adjunctive agent is administered at a dose that is less than a dosage of the at least one adjunctive agent required to provide a therapeutically efficacious plasma level of the at least one adjunctive agent when administered alone.

34. The method of claim 27 or 28, wherein the at least one adjunctive agent is selected from the group consisting of a mood stabilizer, an anticonvulsant, an antipsychotic, an anxiolytic, and a cholinergic agonist.

35. The method of claim 34, wherein the cholinergic agonist is selected from the group consisting of donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof.

36. The method of claim 35, wherein the cholinergic agonist is carbachol and pharmaceutically acceptable salts thereof.

37. The method of claim 35, wherein the cholinergic agonist is donepezil and pharmaceutically acceptable salts thereof.

38. The method of claim 34, wherein the antipsychotic is selected from the group consisting of haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof.

39. The method of claim 38, wherein said antipsychotic is clozapine and pharmaceutically acceptable salts thereof.

40. The method of claim 36, wherein the carbachol is administered to said patient to provide a plasma concentration of 10 μM or less.

41. The method of claim 37, wherein the donepezil is administered to said patient to provide a plasma concentration of 10 ng/ml or less.

42. The method of claim 39, wherein the clozapine is administered to said patient to provide a plasma concentration of 100 ng/ml or less.

43. A pharmaceutical combination comprising a lithium compound and at least one adjunctive agent.

44. A pharmaceutical composition comprising a lithium compound and at least one adjunctive agent; and a pharmaceutically acceptable carrier.

45. The pharmaceutical combination or composition of claim 43 or 44, wherein the effective amount of lithium compound is a dose amount that is less than a dosage of lithium required to provide a therapeutically efficacious plasma lithium level for BD therapy when used alone.

46. The pharmaceutical combination or composition of claim 45, wherein the dose provides a plasma lithium level of 1 mM or less.

47. The pharmaceutical combination or composition of claim 46, wherein the dose provides a plasma lithium level of 0.5 mM or less.

48. The pharmaceutical combination or composition of claim 43 or 44, wherein the at least one adjunctive agent is administered at a dose that is less than a dosage of the at least one adjunctive agent required to provide a therapeutically efficacious plasma level of the at least one adjunctive agent when administered alone.

49. The pharmaceutical combination or composition of claim 43 or 44, wherein the at least one adjunctive agent is selected from the group consisting of a mood stabilizer, an anticonvulsant, an antipsychotic, an anxiolytic, and a cholinergic agonist.

50. The pharmaceutical combination or composition of claim 49, wherein the cholinergic agonist is selected from the group consisting of donepezil, galantamine, rivastigmine, tacrine, donepezil/memantine, methoctramine, AF-DX384, acetylcholine, methacholine, arecoline, bethanechol, carbachol, pilocarpine, muscarine, cevimeline, nicotine, and pharmaceutically acceptable salts thereof.

51. The pharmaceutical combination or composition of claim 50, wherein the cholinergic agonist is carbachol and pharmaceutically acceptable salts thereof.

52. The pharmaceutical combination or composition of claim 50, wherein the cholinergic agonist is donepezil and pharmaceutically acceptable salts thereof.

53. The pharmaceutical combination or composition of claim 49, wherein the antipsychotic is selected from the group consisting of haloperidol, loxapine, thioridazine, molindone, thiothixene, fluphenazine, mesoridazine, trifluoperazine, perphenazine, chlorpromazine, aripiprazole, clozapine, ziprasidone, risperidone, asenapine, cariprazine, olanzapine, quetiapine, lurasidone, olanzapine, loxapine, and pharmaceutically acceptable salts thereof.

54. The pharmaceutical combination or composition of claim 53, wherein said antipsychotic is clozapine and pharmaceutically acceptable salts thereof.

55. The pharmaceutical combination or composition of claim 51, wherein the carbachol is administered to said patient to provide a plasma concentration of 10 μM or less.

56. The pharmaceutical combination or composition of claim 52, wherein the donepezil is administered to said patient to provide a plasma concentration of 10 ng/ml or less.

57. The pharmaceutical combination or composition of claim 54, wherein the clozapine is administered to said patient to provide a plasma concentration of 100 ng/ml or less.

58. A kit comprising:
(a) a reference buffer;
(b) a test buffer;
(c) a potential-sensitive dye; and
(d) instructions for performing an assay to determine an optimal combination drug treatment therapy for bipolar disorder.

59. A kit comprising:
(a) a reference buffer;
(b) a test buffer;
(c) a potential-sensitive dye; and
(d) instructions for performing an assay to optimize a combination drug treatment therapy for bipolar disorder.

60. A kit comprising:
(a) a reference buffer;
(b) a test buffer;
(c) a potential-sensitive dye; and
(d) instructions for performing an assay to determine an optimum dosage of a drug in combination drug treatment therapy for bipolar disorder.

61. A kit comprising:
(a) a reference buffer;
(b) a test buffer,
(c) a potential-sensitive dye; and
(d) instructions for performing an assay to monitor the efficacy of a combination drug treatment therapy for bipolar disorder.

62. The kit of claim 58, 59, 60, or 61, wherein the reference buffer contains NaCl, Cacl2, glucose and hepes.

63. The kit of claim 58, 59, 60, or 61, wherein the test buffer contains ethyl alcohol, NaCl, Cacl2, glucose and hepes.
